SEQUENCE LISTING (17 sequences)

SEQ ID NO: 1 - protein, 8 aa, Artificial Sequence
  Artificial Peptide
FIEWLKNG

SEQ ID NO: 2 - protein, 4 aa, Artificial Sequence
  Artificial Peptide
PTGS

SEQ ID NO: 3 - protein, 12 aa, Artificial Sequence
  Artificial Peptide
FIEWLKNGPTGS

SEQ ID NO: 4 - protein, 29 aa, Artificial Sequence
  Artificial Peptide
SKQMEEEAVRLFIEWLKNGGPSSGAPPPS

SEQ ID NO: 5 - protein, 10 aa, Artificial Sequence
  Artificial Peptide
HGEGTFTSDL

SEQ ID NO: 6 - protein, 39 aa, Artificial Sequence
  Artificial Peptide
HGEGTFTSDLSKQMEEEAVRLFIEWLKNGGPSSGAPPPS

SEQ ID NO: 7 - protein, 5 aa, Artificial Sequence
  Artificial Peptide
PILPP

SEQ ID NO: 8 - protein, 4 aa, Artificial Sequence
  Artificial Peptide
EEYL

SEQ ID NO: 9 - protein, 9 aa, Artificial Sequence
  Artificial Peptide
PILPPEEYL

SEQ ID NO: 10 - protein, 10 aa, Artificial Sequence
  Artificial Peptide
FPILPPEEYL

SEQ ID NO: 11 - protein, 11 aa, Artificial Sequence
  Artificial Peptide
SFPILPPEEYL

SEQ ID NO: 12 - protein, 4 aa, Artificial Sequence
  Artificial Peptide
GGGG

SEQ ID NO: 13 - protein, 15 aa, Artificial Sequence
  Artificial Peptide
GGGGSFPILPPEEYL

SEQ ID NO: 14 - protein, 16 aa, Artificial Sequence
  Artificial Peptide
GGGGGSFPILPPEEYL

SEQ ID NO: 15 - protein, 4 aa, Artificial Sequence
  Artificial Peptide
AGFS

SEQ ID NO: 16 - protein, 5 aa, Artificial Sequence
  Artificial Peptide
PAGFS

SEQ ID NO: 17 - protein, 5 aa, Artificial Sequence
  Artificial Peptide
LWVNS

(19) United States
(12) Patent Application Publication
(10) Pub. No.: US 2014/0128572 A1
(43) Pub. Date: May 8, 2014

(54) Process For Extraction Of Peptides And Its Application In Liquid Phase Peptide Synthesis

(71) Applicants: Didier Monnaie, Houdeng Aimeries (BE); Luciano Forni, La Louviere (BE); Mathieu Giraud, Sion (CH)

(72) Inventors: Didier Monnaie, Houdeng Aimeries (BE); Luciano Forni, La Louviere (BE); Mathieu Giraud, Sion (CH)

(73) Assignees: Lonza Braine S.A., Braine l'Alleud (BE); Lonza Ltd, Visp (CH)

(21) Appl. No.: 14/125,804

(22) PCT Filed: Jun. 14, 2012
(86) PCT No.: PCT/EP2012/061257

§ 371 Date: Jan. 24, 2014

Related U.S. Application Data
(60) Provisional application No. 61/498,100, filed on Jun. 17, 2011.
(60) Provisional application No. 61/497,642, filed on Jun. 16, 2011.

(30) Foreign Application Priority Data
Jun. 16, 2011 (EP) 11170094.4

(51) Int. Cl. C07K 1/14

(52) U.S. Cl. CPC C07K 1/145
USPC 530/338; 530/344

(57) ABSTRACT

A process for extraction of a peptide from a reaction mixture resulting from a peptide coupling reaction, the reaction mixture containing the peptide and a polar aprotic solvent selected from N,N-dimethylformamide, N,N-dimethylacetamide and N-methyl-2-pyrrolidone, whereby the process includes a step a) and a step b): step a) including the addition of a component a1), a component a2) and a component a3), whereby component a1) is an organic solvent 1, the organic solvent 1 is selected from 2-methyltetrahydrofuran and toluene, component a2) is water, and component a3) is an organic solvent 2, the organic solvent 2 is selected from the ethylacetate, isopropylacetate, acetonitrile, tetrahydrofuran and n-heptane to the reaction mixture, so that a biphasic system with an organic layer and an aqueous layer is obtained; step b) including the subsequent separation of the organic layer containing the peptide from the aqueous layer. The extraction step is preferably used in a process for preparation of a peptide in liquid phase.

### FIELD OF THE INVENTION

The present invention relates to a process for extraction of a peptide from a reaction mixture resulting from a peptide coupling reaction. This process is preferably used in a method of liquid phase peptide synthesis (LPPS). The process for extraction of a peptide from a reaction mixture can also be used in other types of peptide synthesis, for example in a postcleavage isolation of synthetic peptides prepared by a solid phase peptide synthesis (SPPS). This process is also applicable for hybrid solid and liquid phase peptide synthesis. Moreover, the process for extraction of a peptide can be employed for the isolation of peptides from natural sources such as yeast or bacteria, in particular for the isolation of recombinantly expressed peptides.

### BACKGROUND OF THE PRESENT INVENTION

In the text of the present application, the nomenclature of amino acids and of peptides is used according to “Nomenclature and symbolism for amino acids and peptides”, Pure & Appl. Chem. 1984, Vol. 56, No. 5, pp. 595-624, if not otherwise stated.

The following abbreviations have the meaning as given in the following list, if not otherwise stated:
ACN acetonitrile
Boc tert-butoxycarbonyl
Bsmoc 1,1-dioxobenzo[b]thiophen-2-ylmethyloxycarbonyl
Bzl benzyl
Cbz benzyloxycarbonyl
DCC N,N-dicyclohexylcarbodiimide
DCE dichloroethane
DCM dichloromethane
DCU N,N-dicyclohexylurea
DEA diethylamine
DIPE diisopropyl ether
DIPEA N,N-diisopropylethylamine
DMA N,N-dimethylacetamide
DMF N,N-dimethylformamide
DOE design of experiments
EDC 1-ethyl-3-(3-dimethylaminopropyl)carbodiimide
eq equivalent(s)
EtOAc ethylacetate
Fmoc fluorenyl-9-methoxycarbonyl
h hour(s)
HOBt 1-hydroxybenzotriazole
HOBt.H₂O 1-hydroxybenzotriazole monohydrate
HPLS high-performance liquid chromatography
LPPS liquid phase peptide synthesis
MeTHF 2-methyltetrahydrofuran
min minute(s)
MS mass spectrometry
NMP N-methyl-2-pyrrolidone
OMe methoxy
OtBu tert-butoxy
PG protecting group
PyBOP benzotriazol-1-yloxy-tris(pyrrolidino)-phosphonium hexafluorophosphate
RM reaction mixture
SPPS solid phase peptide synthesis TAEA tris(2-aminoethyl)amine
TBTU O-(benzotriazol-1-yl)-1,1,3,3-tetramethyluronium tetrafluoroborate
tBu tert-butyl
TEA triethylamine
TFA trifluoroacetic acid
THF tetrahydrofuran
TLC thin layer chromatography
TOTU O-[cyano(ethoxycarbonyl)methylenamino]-1,1,3,3-tetramethyluronium tetrafluoroborate
Trt trityl
UV ultraviolet

Processes for extraction of peptides are generally employed in various types of peptide synthesis, such as liquid phase peptide synthesis (LPPS), solid phase peptide synthesis (SPPS) as well as hybrid solid and liquid phase peptide synthesis.

LPPS is particularly often used for industrial large-scale preparations of peptides. LPPS typically involves coupling of two partially protected amino acids or peptides, whereby one of them bears an unprotected C-terminal carboxylic acid group and the other one bears an unprotected N-terminal amino group. After completion of the coupling step, the N-terminal amino group or, alternatively, the C-terminal carboxylic acid group of the resulting peptide can be deprotected by specific cleavage of one of its protecting groups (PGs), so that a subsequent coupling step can be carried out. LPPS is usually finalised by a global deprotection step, in which all remaining PGs are removed.

The handling of peptides, in particular of peptides bearing an unprotected C-terminal carboxylic acid group and/or an unprotected N-terminal amino group during the LPPS, is often compromised by the poor solubility of the peptides in common organic solvents. In general, the solubility of peptides in common organic solvents decreases with the length of the peptide chain.

Dichloromethane (DCM) is commonly used in LPPS as a suitable reaction solvent. DCM has good solvent properties, a low boiling point and its limited miscibility with water allows working-up of the reaction mixtures by extraction with an aqueous solution. The use of DCM on an industrial scale is, however, problematic for environmental reasons and generally limited due to its high density, which makes an extraction of a DCM layer with an aqueous solution time and cost-consuming.

Furthermore, some recently developed and highly efficient coupling reagents such as benzotriazol-1-yloxy-tris(pyrrolidino)-phosphonium hexafluorophosphate (PyBOP) and O-(benzotriazol-1-yl)-1,1,3,3-tetramethyluronium tetrafluoroborate (TBTU) are poorly soluble in DCM. These coupling reagents are particularly advantageous for a coupling of two large peptide fragments, which is known to be low-yielding upon usage of other coupling reagents.

In addition, many peptides show only a poor solubility in DCM under neutral and basic conditions and are only sufficiently soluble in polar aprotic solvents, such as e.g. N,N-dimethylformamide (DMF), N,N-dimethylacetamide (DMA) or N-methyl-2-pyrrolidone (NMP). Therefore, these polar aprotic solvents are traditionally used as reaction solvents in LPPS, alone or in a mixture with a less polar solvent such as tetrahydrofuran (THF).

On the other hand, the usage of polar aprotic solvents for LPPS suffers from a number of drawbacks. Since polar aprotic solvents have a high boiling point, it is difficult to concentrate the reaction mixture by evaporation. Furthermore, a direct working-up of the reaction mixture by extraction with an aqueous solution is not possible due to the miscibility of polar aprotic solvents with water.

When LPPS is carried out on an industrial scale, the intermediate peptide is usually isolated by a direct precipitation from the reaction mixture after each coupling step, so that impurities, such as unreacted starting materials, side products as well as an excess of coupling reagents and bases, etc. can be separated. After the completion of the peptide coupling reaction, the reaction mixture is typically poured into an anti-solvent, such as e.g. diethyl ether or water, whereby the precipitation of the peptide takes place. Unfortunately, already the transfer of the reaction mixture into the anti-solvent is known to trigger gel formation issues.

Moreover, polar aprotic solvents commonly interfere with the process of peptide precipitation, so that the precipitated peptide is obtained as a sticky gum-like solid, which is difficult to filter and to dry. In some cases, it is not possible to filter the precipitated peptide or not even possible to transfer the precipitated peptide onto a filter. Particularly, peptide precipitations carried out on an industrial scale are often difficult to perform and are very time-consuming, whereby the filtration time determines the lead time. This problem can be partially overcome by an increase of the volume ratio anti-solvent:polar aprotic solvent during the precipitation process, so that in practice a large amount of a suitable anti-solvent is required for obtaining the precipitated peptide in a filterable form.

In addition, residues of polar aprotic solvents present in the precipitated peptide are known to interfere with the subsequent deprotection step involving trifluoroacetic acid (TFA). Therefore, an additional step of removal of the polar aprotic solvent residues by washing the precipitated peptide with a more volatile solvent is necessary before a cleavage of acid cleavable type PGs such as tert-butoxycarbonyl (Boc), trityl (Trt), tert-butyl (tBu) and tert-butoxy (OtBu) can be carried out.

### DESCRIPTION OF RELATED ART

WO 2005/081711 is directed to drug-linker-ligand conjugates and drug-linker compounds and to methods for using the same to treat cancer, an autoimmune disease or an infectious disease. The document discloses inter alia methods for preparation of peptide based drugs and extractions of peptides using ethylacetate, dichloromethane and a mixture of tBuOH/CHCl₃.

U.S. Pat. No. 5,869,454 is directed to arginine keto-amide enzyme inhibitors. The document discloses inter alia synthesis of these inhibitors and extractions with ethylacetate.

US 2005/0165215 relates to methods of synthesizing peptides and methods for the isolation of peptides during the synthetic process. The document further relates to improvements for the large scale synthesis of peptides. The document suggests that suitable solvents for the peptide extractions include halogenated organic solvents, such as dichloropropane, dichloroethane, dichloromethane, chloroform, chlorofluorocarbons, chlorofluorohydrocarbons and mixtures thereof. A preferred solvent is dichloromethane.

C. H. Schneider et al. (Int. J. Peptide Protein Res. 1980, 15, pp. 411-419) describes a procedure of peptide synthesis in solution based on liquid-liquid extraction for the purification of intermediates (two-phase method). The peptide extractions employ dichloromethane as a solvent.

J. W. van Nispen (Pure and Appl. Chem. 1987, Vol. 59, No. 3, pp. 331-344,) provides an overview over synthesis and analysis of (poly)peptides. The document teaches that a large number of combinations of solvents of widely varying nature is possible in order to find optimal separation of peptide components. For this purpose so-called Craig machines are commonly employed, where in the multiplicative distribution, the lower phase retains its position while the upper phase is mobile.

US 2010/0184952 discloses a method of removing dibenzofulvene and/or a dibenzofulvene amine adduct from a reaction mixture obtained by reacting an amino acid compound protected with an Fmoc group with an amine for deprotection, which comprises stirring and partitioning the reaction mixture in a hydrocarbon solvent having a carbon number of 5 or above and a polar organic solvent (excluding organic amide solvents) immiscible with the hydrocarbon solvent, and removing the hydrocarbon solvent layer in which the dibenzofulvene and/or the dibenzofulvene amine adduct are/is dissolved. During this method, an amino acid ester or peptide is transferred to a polar organic solvent. Examples of such polar organic solvents include acetonitrile, methanol, acetone and the like and a mixed solvent thereof, with preference given to acetonitrile and methanol.

L. A. Carpino et al. (Organic Process Research & Development 2003, 7, pp. 28-37) describe a rapid, continuous solution-phase peptide synthesis. The methods employing deprotections of the Fmoc and Bsmoc protective groups of peptide segments in the presence of tris(2-aminoethyl)amine were shown to be applicable for the gram-scale rapid, continuous solution synthesis of short peptides as well as for the synthesis of a relatively long (22-mer) segment (hPTH 13-34). In the latter case, the crude product was reported to be of a significantly greater purity than a sample obtained via a solid-phase protocol. The Bsmoc methodology was optimised by a new technique involving filtration of the growing partially deprotected peptide at each coupling deprotection cycle through a short column of silica gel.

However, the methodology described by L. A. Carpino et al. has several limitations. This methodology employs DCM as a reaction solvent and, therefore, cannot be applied for the preparation of peptides showing a poor solubility in DCM. Moreover, it employs a high quantity of high-cost tris(2-aminoethyl)amine (TAEA) which further limits the applicability of this methodology on an industrial scale.

Thus, there is a strong demand for a time- and cost-efficient synthetic methodology for the preparation of peptides, in particular on an industrial scale. Such methodology must overcome the drawbacks resulting from the usage of DCM and of polar aprotic solvents such as DMF, DMA and NMP during LPPS.

### SUMMARY OF THE INVENTION

The authors of the present invention surprisingly found that a broad range of structurally diverse peptides has an excellent solubility in a combination of an organic solvent selected from the group consisting of 2-methyltetrahydrofuran and toluene (this group is designated as organic solvent 1) and with an organic solvent selected from the group consisting of ethylacetate, isopropylacetate, acetonitrile, n-heptane and tetrahydrofuran (this group is designated as organic solvent 2). In particular, the solubility of the peptides in the combination of the organic solvent 1 and the organic solvent 2 is significantly higher than in the neat organic solvent 1. Moreover, they found that commonly used polar aprotic solvents largely partition into the aqueous layer in a biphasic system comprising water and a combination of the organic solvent 1 and the organic solvent 2.

Therefore, a combination of the organic solvent 1 with the organic solvent 2 and water is highly suitable for the extraction of a peptide from a mixture containing a polar aprotic solvent. In one of the embodiments of the present invention, the resulting organic layer containing the peptide is partially evaporated and the peptide dissolved therein is precipitated upon addition of a suitable anti-solvent (this group is designated as organic solvent 3). Because substantially no polar aprotic solvent is present during the process of peptide precipitation the resulting peptide can easily be filtered. By applying the extraction process of the present invention, the time required for the peptide filtration can be significantly reduced. Thus, by applying such process of extraction, the drawbacks resulting from the usage of polar aprotic solvents during LPPS can be successfully overcome.

The present invention relates to a process for extraction of a peptide from a reaction mixture resulting from a peptide coupling reaction, the reaction mixture containing the peptide and a polar aprotic solvent selected from the group consisting of N,N-dimethylformamide, N,N-dimethylacetamide and N-methyl-2-pyrrolidone, whereby the process comprises a step a) and a step b):

step a) comprises the addition of a component a1), a component a2) and a component a3) whereby

component a1) is an organic solvent 1, the organic solvent 1 is selected from the group consisting of 2-methyltetrahydrofuran and toluene,

component a2) is water, and component a3) is an organic solvent 2, the organic solvent 2 is selected from the group consisting of ethylacetate, isopropylacetate, acetonitrile, tetrahydrofuran and n-heptane

to the reaction mixture, so that a biphasic system with an organic layer and an aqueous layer is obtained;

step b) comprises the separation of the organic layer containing the peptide from the aqueous layer, whereby

the biphasic system obtained in step a) is characterised by the following volume ratios:

polar aprotic solvent:organic solvent 1 from 1:20 to 1:2;

polar aprotic solvent:organic solvent 2 from 1:5 to 30:1; and

polar aprotic solvent:water from 1:20 to 1:2.

In a preferred embodiment, the biphasic system obtained in step a) is characterised by the following volume ratios:

polar aprotic solvent:organic solvent 1 from 1:6 to 1:3;

polar aprotic solvent:organic solvent 2 from 1:1 to 4:1;

polar aprotic solvent:water from 1:5 to 1:3; and

organic solvent 1:organic solvent 2 from 10:1 to 2:1.

In a particularly preferred embodiment, the polar aprotic solvent is N,N-dimethylformamide or N-methyl-2-pyrrolidone.

In a preferred embodiment of the present invention, the organic solvent 1 is 2-methyltetrahydrofuran.

In one of the preferred embodiments of the present invention, the peptide is extracted but not precipitated. Instead, one or several protecting groups of the peptide are cleaved and the resulting partially unprotected peptide is extracted and the organic layer comprising the peptide is employed for the subsequent peptide coupling reaction. Thus, the present invention provides an efficient synthetic methodology for a continuous LPPS which is suitable for the preparation of peptides on an industrial scale.

The continuous LPPS of the present invention is highly suitable for the peptide synthesis upon usage of Boc, Fmoc and Bzl as protective groups as will be illustrated by the examples below.

### Process for Extraction

The current invention relates to a process for extraction of a peptide from a reaction mixture resulting from a peptide coupling reaction containing the peptide and a polar aprotic solvent selected from the group consisting of DMF, DMA and NMP, whereby the process comprises a step a) and a step b):

step a) comprises the addition of a component a1), a component a2) and a component a3), whereby

component a1) is an organic solvent 1, the organic solvent 1 is selected from the group consisting of 2-methyltetrahydrofuran and toluene,

component a2) is water,

component a3) is an organic solvent 2, the organic solvent 2 is selected from the group consisting of ethylacetate, isopropylacetate, acetonitrile, tetrahydrofuran and n-heptane,

so that a biphasic system with an organic layer and an aqueous layer is obtained;

step b) comprises the separation of the organic layer containing the peptide from the aqueous layer.

Optionally, the component a1), the component a2) and the component a3) are mixed with each other, whereby this can be done in any sequence. The three components can also be added as premixed mixtures of two or all three components as long as no precipitation of the peptide takes place during the process for extraction.

The mixture containing the polar aprotic solvent is preferably a crude reaction mixture resulting from a peptide coupling reaction. Preferably, this mixture does not contain any compounds, which can act as surfactants and interfere with the phase separation during the process for extraction. In a particularly preferred embodiment the mixture does not contain any surfactants known in the prior art, such as cationic tensides and non-ionic tensides.

The addition of the component a1), the component a2) and the component a3) to the mixture containing the peptide and a polar aprotic solvent can take place in any order as long as no precipitation of the peptide takes place during the process for extraction. For example, it is possible to combine the mixture containing the peptide and a polar aprotic solvent with the organic solvent 1, add water thereto and, finally, add the organic solvent 2. It is also possible that the mixture containing the peptide and a polar aprotic solvent is transferred into the water and the organic solvent 1 and the organic solvent 2 are added thereto afterwards.

In the particularly preferred embodiment of the present invention, the mixture containing the peptide and a polar aprotic solvent is combined with the organic solvent 1 and the organic solvent 2, whereby the addition of the organic solvent 1 and the organic solvent 2 can take place in any order. Subsequently, water is added thereto.

It is understood that the added water (component a2)) may contain dissolved components, such as salts, for instance inorganic salts.

It is preferred that the obtained biphasic system is vigorously stirred. The process of stirring of the obtained biphasic system can be carried out upon usage of mixing equipment known in the state of the art and commonly used for extractions. For example, in the case of batch extractions, jet- or agitator-type mixers can be employed for the stirring of the biphasic system.

The choice of the suitable equipment for the extraction mainly depends on the scale on which the process for extraction is being carried out as well as on the extraction temperature. The process for extraction can be carried out by using batch extractions or continuous extractions. The process for extraction can also be repeated several times, if required, so that an optimal extraction of the peptide is achieved.

After the process of stirring has been carried out, it is preferred that a phase separation is allowed to take place, whereby two liquid layers are formed: an organic layer and an aqueous layer. The organic layer has a lower density than the aqueous layer. Phase separation may be accomplished upon usage of settling tanks or by means of centrifugation. The time required for the phase separation depends on the scale on which the process for extraction is taking place and on the equipment employed. Preferably, the phase separation requires less than 1 hour, more preferred less than 10 min, particularly preferred less than 1 min.

After the phase separation has taken place, the peptide is mainly located in the organic layer, which further contains the organic solvent 1 and the organic solvent 2. The upper organic layer containing the peptide is separated from the aqueous layer. Preferably, after the process for extraction more than 90 wt.-% of the peptide is located in the organic layer and less than 10 wt.-% of the peptide is located in the aqueous layer. It is even more preferred that after the process for extraction more than 98 wt.-% of the peptide is located in the organic layer and less than 2 wt.-% of the peptide is located in the aqueous layer. It is particularly preferred that after the process for extraction more than 99 wt.-% of the peptide is located in the organic layer and less than 1 wt.-% of the peptide is located in the aqueous layer.

The process for extraction of the present invention allows an efficient extraction of the peptide from a crude reaction mixture resulting from a peptide coupling reaction. The solubility of polar aprotic solvents in the organic layer is significantly lower than in the aqueous layer. Therefore, the organic layer containing the peptide further contains only a low amount of the polar aprotic solvents after the extraction.

Preferably, after the process for extraction less than 15 vol.-% of the polar aprotic solvents is located in the organic layer and more than 85 vol.-% of the polar aprotic solvents is located in the aqueous layer. It is, however, more preferred that after the process for extraction less than 5 vol.-% of the polar aprotic solvents is located in the organic layer and more than 95 vol.-% of the polar aprotic solvents is located in the aqueous layer. It is particularly preferred that after the process for extraction less than 2 vol.-% of the polar aprotic solvents is located in the organic layer and more than 98 vol.-% of the polar aprotic solvents is located in the aqueous layer. This may require repeated extractions.

Importantly, the process for extraction according to the present invention not only allows to separate the peptide from a substantial part of the polar aprotic solvent but also from salts and side products, which originate from the coupling reagents (ureas, tetrafluoroborates etc.). These salts and side products usually cannot be removed if a direct precipitation from a crude reaction mixture resulting from a peptide coupling reaction takes place upon addition of a hydrophobic anti-solvent such as n-heptane or diethyl ether. However, these salts and side products are known to reduce the capacity of chromatography columns used for the downstream processing of peptides. Such additional purification by column chromatography is essential if the prepared peptides are used as active pharmaceutical ingredients.

Thus, if required, the precipitated peptide can be subsequently purified by column chromatography. In cases wherein the peptide is used as an active pharmaceutical ingredient such additional purification steps are used. Therefore, the process for extraction according to the present invention allows isolating the peptide in a higher purity than upon usage of the direct precipitation process from the reaction mixture.

The composition of the biphasic system obtained during the process for extraction has a strong impact on the distribution coefficients of the peptide and of the polar aprotic solvents between the organic layer and the aqueous layer. In the following the ratios are given as volume to volume ratios.

It is preferred that the volume ratio polar aprotic solvent:organic solvent 1 ranges from 1:20 to 1:2. Preferably, this volume ratio ranges from 1:10 to 1:2. It is particularly preferred that this volume ratio ranges from 1:6 to 1:3.

The solubility of the peptide in a combination of the organic solvent 1 and the organic solvent 2 was shown to be higher than in the neat organic solvent 1. Therefore, the solubility of the peptide in the organic layer obtained during the process for extraction is particularly high when the amount of the organic solvent 2 used is sufficiently high. It is preferred that the volume ratio polar aprotic solvent:organic solvent 2 ranges from 1:5 to 30:1. Preferably, this volume ratio ranges from 1:3 to 10:1. It is particularly preferred that this volume ratio ranges from 1:1 to 4:1.

It is preferred that the volume ratio organic solvent 1:organic solvent 2 ranges from 50:1 to 1:1. Preferably, this volume ratio ranges from 20:1 to 2:1. It is particularly preferred that this volume ratio ranges from 10:1 to 2:1.

The volume ratio polar aprotic solvent:water has a significant influence on the efficiency of the process for extraction and on the solubility of the peptide in the aqueous layer. In particular, the peptide has a considerably high solubility in the aqueous layer, if the volume ratio polar aprotic solvent:water in the biphasic system is higher than 1:2, e.g. if the aqueous layer contains more than 34 vol.-% of the polar aprotic solvent. It is therefore preferred that the volume ratio polar aprotic solvent:water ranges from 1:20 to 1:2. Preferably, this volume ratio ranges from 1:10 to 1:3. It is particularly preferred that this volume ratio ranges from 1:5 to 1:3.

Preferably, the polar aprotic solvent present in the mixture containing the peptide is selected from the group consisting of DMF and NMP.

It has been found that a combination of the organic solvent 1 and the organic solvent 2 is particularly suitable for the process for extraction of a peptide if the organic solvent 1 is 2-methyltetrahydrofuran. Thus, the organic solvent 1 used in the process for extraction is preferably 2-methyltetrahydrofuran. 2-Methyltetrahydrofuran is an easily recyclable, environmentally friendly solvent, which can be derived from a variety of agricultural by-products. Accordingly, the present invention provides an environmentally friendly process for extraction of a peptide.

The solubility of the peptide in a combination of the organic solvent 1 and the organic solvent 2 is particularly high if the organic solvent 2 is selected from the group consisting of ethylacetate (EtOAc), isopropylacetate, acetonitrile (ACN), n-heptane and tetrahydrofuran (THF), more preferred from the group consisting of EtOAc, isopropylacetate, ACN and THF, particularly preferred from the group consisting of ACN and THF. Accordingly, in a particularly preferred embodiment for the process for extraction of the peptide, the organic solvent 2 is selected from the group consisting of ACN and THF.

The component a2) employed for the process for extraction of the peptide can consist of water only. However, the miscibility of the organic solvent 1 and of the organic solvent 2 with the component a2) and, consequently, the solubility of the peptide in the aqueous layer can be significantly reduced if the component a2) further contains at least one inorganic salt. In addition, the water content in the organic layer is reduced if the component a2) contains at least one inorganic salt.

In one of the preferred embodiments the component a2) contains at least one inorganic salt selected from the group consisting of sodium chloride, sodium hydrogensulfate, potassium hydrogensulfate, sodium hydrogencarbonate and sodium hydrogenphosphate. In other embodiments the component a2) can also contain other compounds such as acids.

In particular, the component a2) can contain inorganic salts which do not act as buffering agents in the pH range from 2 to 11. An addition of such inorganic salts can decrease the solubility of the peptide in the aqueous layer and reduce the time required for the phase separation during the process for extraction. For instance, the component a2) can contain sodium chloride or sodium sulfate. The concentration of the inorganic salt present in the component a2) preferably ranges from 1 wt.-% to 20 wt.-%, even more preferred from 5 wt.-% to 15 wt.-%. A salt like sodium chloride is used to facilitate the separation of the two phases and a salt that acts as a buffering agent is used to selectively extract an acid or a base in the aqueous layer.

The pH value of the component a2) can have a strong influence on the solubility of the peptide as well as on the solubility of some impurities in the aqueous layer. In addition, the choice of the pH value of the component a2) depends on the chemical stability of the peptide as well as on the chemical stability of its PGs. It is preferred that the pH value of the component a2) ranges from 2 to 11, particularly preferred from 5 to 8, so that the tertiary bases used for the peptide coupling reaction predominantly remain in the aqueous layer during the process for extraction. The pH value of the component a2) can be adjusted by an addition of an acid or a base and/or upon using a buffering agent.

The choice of the acid which can be used for the adjustment of the pH value of the component a2) is not particularly limited as long as the acid present in the component a2) does not interfere with the process for extraction of the peptide and does not cause the degradation of the peptide. For example, Brønsted acids such as sulphuric acid, hydrochloric acid, phosphoric acid, trifluoroacetic acid or citric acid can be employed for this purpose.

The choice of the base which can be used for the adjustment of the pH value of the component a2) is not particularly limited as long as the base present in the component a2) does not interfere with the process for extraction of the peptide and does not cause the degradation of the peptide. For example, hydroxides of alkali metals such as sodium hydroxide, potassium hydroxide and lithium hydroxide are suitable for the adjustment of the pH value of the component a2).

It is preferred that the component a2) contains the buffering agent, so that the pH value of the aqueous layer is kept within the desired range during the process for extraction. Preferably, the buffering agent is selected from the group consisting of ammonium chloride, sodium hydrogensulfate, potassium hydrogensulfate, sodium hydrogencarbonate, sodium carbonate, sodium hydrogenphosphate, sodium dihydrogenphosphate and sodium phosphate. The concentration of the buffering agent present in the component a2) preferably ranges from 1 wt.-% to 10 wt.-%, even more preferred from 3 wt.-% to 8 wt.-%.

Optionally, the obtained organic layer containing the peptide can be additionally washed at least one time with an aqueous solution. Preferably, the pH value of the aqueous solution used for this purpose ranges from 2 to 11.

Depending on the conditions of the peptide coupling reaction and the reagents used, the organic layer can contain compounds with free primary, secondary or tertiary amino groups as impurities, for instance, peptides with unprotected N-terminal amino groups or tertiary bases. In such cases, it is preferred that the organic layer is washed with an aqueous solution having a pH value of from 2 to 7.

In other cases, the organic layer can contain compounds having a free carboxylic acid group, for instance, peptides with unprotected C-terminal carboxylic acid groups. In these cases, it is preferred that the organic layer is washed with an aqueous solution having a pH value of from 7 to 11.

The temperature at which the process for extraction of the peptide is preferably carried out (hereinafter designated as extraction temperature) depends on the choice of the solvents employed as well as on the properties of the peptide. The extraction temperature has a strong influence on the miscibility of the solvents employed and on the solubility of the peptide in the organic layer and in the aqueous layer. The extraction temperature is therefore chosen in such a way that a biphasic system is formed during the process for extraction and the solubility of the peptide in the organic layer is sufficiently high. Preferably, the process for extraction of the peptide is carried out at the extraction temperature of from 0° C. to 60° C. It is particularly preferred that the extraction temperature ranges from 20° C. to 30° C.

Depending on the conditions of the peptide coupling reaction and on the coupling reagents employed, a formation of solids can take place before and/or during the process for extraction. This can be, for instance, the case, if carbodiimides are used as coupling reagents. For this reason, it may be required that a filtration of the biphasic system obtained after combining the mixture containing the peptide and a polar aprotic solvent with the organic solvent 1, the organic solvent 2 and the component a2) is carried out. Therefore, in one of the embodiments of the present invention a filtration of the biphasic system is carried out before the organic layer containing the peptide is separated.

The peptide extracted by the process for extraction of the present invention may be any peptide. Preferably, the peptide extracted by the process for extraction comprises 100 or less amino acid residues, more preferably 50 or less amino acid residues, most preferably 20 or less amino acid residues. The amino acids of the peptide can be D- and/or L-α-amino acids, β-amino acids as well as other organic compounds containing at least one primary and/or secondary amino group and at least one carboxylic acid group. Preferably, the amino acids are α-amino acids, even more preferably L-α-amino acids, whereby proteinogenic amino acids are particularly preferred.

### Preparation of the Peptide

Another aspect of the present invention relates to a process for preparation of a peptide in liquid phase comprising a step aa), a step bb) and a step cc):

in step aa) a peptide coupling reaction is carried out in the polar aprotic solvent selected from the group consisting of N,N-dimethylformamide, N,N-dimethylacetamide and N-methyl-2-pyrrolidone in the presence of a coupling reagent and, optionally, a tertiary base;

in step bb) the resulting peptide is extracted according to a process described above; and

in step cc) at least a part of the organic layer obtained in step bb) is evaporated.

As starting materials for the peptide coupling reaction according to step aa) a combination of two partially protected amino acids, of two partially protected peptides or a combination of a partially protected amino acid and a partially protected peptide is employed.

The process for preparation of a peptide in liquid phase according to the present invention is highly suitable in a liquid phase peptide synthesis (LPPS). In one of the embodiments of the present invention, the peptide coupling reaction according to step aa) employs a combination of two partially protected peptides prepared by SPPS. Thus, the process of the present invention allows coupling of peptide fragments and can be used in combination with SPPS.

The peptide coupling reaction according to step aa) is carried out using conventional process parameters and reagents typical for peptide coupling reactions.

The peptide coupling reaction is conventionally carried out in a polar aprotic solvent and upon using one or more coupling reagents, preferably in the presence of one or more coupling additives, and preferably in the presence of one or more tertiary bases.

The coupling reagents used for the peptide coupling reaction are chosen in such a way that they do not react with the polar aprotic solvent under the conditions of the peptide coupling reaction and no substantial epimerisation of the stereogenic centre adjacent to the activated carboxylic acid group takes place. Preferred coupling reagents are therefore phosphonium or uronium salts of O-1H-benzotriazole and carbodiimide coupling reagents.

Phosphonium and uronium salts are preferably selected from the group consisting of
BOP (benzotriazol-1-yl-oxy-tris-(dimethylamino)-phosphonium hexafluorophosphate),
PyBOP (benzotriazol-1-yl-oxy-trispyrrolidinophosphonium hexafluorophosphate),
HBTU (O-(1H-benzotriazole-1-yl)-1,1,3,3-tetramethyluronium hexafluorophosphate),
HCTU (O-(1H-6-chloro-benzotriazole-1-yl)-1,1,3,3-tetramethyluronium hexafluorophosphate),
TCTU (O-(1H-6-chlorobenzotriazole-1-yl)-1,1,3,3-tetramethyluronium tetrafluoroborate),
HATU (O-(7-azabenzotriazol-1-yl)-1,1,3,3-tetramethyluronium hexafluorophosphate),
TATU (O-(7-azabenzotriazol-1-yl)-1,1,3,3-tetramethyluronium tetrafluoroborate),
TBTU (O-(benzotriazol-1-yl)-1,1,3,3-tetramethyluronium tetrafluoroborate),
TOTU (O-[cyano(ethoxycarbonyl)methyleneamino]-1,1,3,3-tetramethyluronium tetrafluoroborate),
HAPyU (O-(benzotriazol-1-yl)oxybis-(pyrrolidino)-uronium hexafluorophosphate),
PyAOP (benzotriazole-1-yl-oxy-tris-pyrrolidinophosphonium hexafluorophosphate),
COMU (1-[(1-(cyano-2-ethoxy-2-oxoethylideneaminooxy)-dimethylamino-morpholinomethylene)]-methanaminium hexafluorophosphate),
PyClock (6-chloro-benzotriazole-1-yl-oxy-tris-pyrrolidinophosphonium hexafluorophosphate), PyOxP (O-[(1-cyano-2-ethoxy-2-oxoethylidene)amino]-oxytri(pyrrolidin-1-yl)-phosphonium hexafluorophosphate) and
PyOxB (O-[(1-cyano-2-ethoxy-2-oxoethylidene)amino]-oxytri(pyrrolidin-1-yl)-phosphonium tetrafluoroborate).

Preferred coupling reagents selected from phosphonium or uronium coupling reagents are TBTU, TOTU and PyBOP.

Carbodiimide coupling reagents are preferably selected from the group consisting of diisopropyl-carbodiimide (DIC), dicyclohexyl-carbodiimide (DCC) and water-soluble carbodiimides (WSCDI) such as 1-ethyl-3-(3-dimethylaminopropyl)-carbodiimide (EDC).

Water-soluble carbodiimides are particularly preferred as carbodiimide coupling reagents, whereby EDC is mostly preferred.

The tertiary base employed in the peptide coupling reaction is preferably compatible with the peptide and with the coupling reagent and does not interfere with the process for extraction by acting as a surfactant.

Preferably, the conjugated acid of said tertiary base used in the peptide coupling reaction has a pKa value from 7.5 to 15, more preferably from 7.5 to 10. Said tertiary base is preferably selected from the group consisting of trialkylamines, such as N,N-diisopropylethylamine (DIPEA) or triethylamine (TEA), further N,N-di-C₁₋₄ alkylanilines, such as N,N-diethylaniline, alkylpyridines, such as collidine (2,4,6-trimethylpyridine), or N—C₁₋₄ alkylmorpholines, such as N-methylmorpholine, with any C₁₋₄ alkyl being identical or different and independently from each other straight or branched C₁₋₄ alkyl. DIPEA, TEA and N-methylmorpholine are particularly preferred as tertiary bases for the peptide coupling reaction.

A coupling additive is preferably a nucleophilic hydroxy compound capable of forming activated esters, more preferably having an acidic, nucleophilic N-hydroxy function wherein N is imide or is N-acyl or N-aryl substituted triazeno, the triazeno type coupling additive being preferably a N-hydroxybenzotriazol derivative (or 1-hydroxybenzotriazol derivative) or a N-hydroxybenzotriazine derivative. Such coupling additives have been described in WO 94/07910 and EP 0 410 182.

Preferred coupling additives are selected from the group consisting of N-hydroxysuccinimide (HOSu), 6-chloro-1-hydroxybenzotriazole (Cl-HOBt), N-hydroxy-3,4-dihydro-4-oxo-1,2,3-benzotriazine (HOOBt), 1-hydroxy-7-azabenzotriazole (HOAt), 1-hydroxybenzotriazole (HOBt) and ethyl-2-cyano-2-hydroxyiminoacetate (CHA). CHA is available under trade name OXYMAPURE®. CHA has proved to be an effective coupling additive as epimerisation of the stereogenic centre of the activated carboxylic acid is suppressed to a higher degree in comparison to benzotriazole-based coupling additives. In addition, CHA is less explosive than e.g. HOBt or Cl-HOBt, so that its handling is advantageous and, as a further advantage, the coupling progress can be visually monitored by a colour change of the reaction mixture. Preferably, HOBt is used as coupling additive for the peptide coupling reaction.

In the preferred embodiment of the present invention, the combination of reagents in the peptide coupling reaction is selected from the group consisting of TBTU/HOBt/DIPEA, PyBOP/TEA, EDC/HOBt and EDC/HOBt/DIPEA.

The reaction solvent for the peptide coupling reaction is selected from the group consisting of DMF, DMA, NMP or mixtures thereof. The particularly preferred reaction solvent for the peptide coupling reaction is selected from the group consisting of DMF and NMP.

Preferably, the reaction solvent is substantially water-free. Preferably, the reaction solvent contains less than 1 wt.-% water, more preferred less than 0.1 wt.-% water, even more preferred less than 0.01 wt.-% water and particularly preferred less than 0.001 wt.-% water. The water content in a solvent can be determined by Karl Fischer titration according to the standard test method ASTM E203-8 as known in the prior art.

Preferably, the reaction solvent for the peptide coupling reaction is substantially free of impurities selected from the group consisting of primary and secondary amines, carboxylic acids and aliphatic alcohols. The reaction solvent for the peptide coupling reaction is considered to be substantially free of these impurities if less than 1 mol.-% of any of the starting materials used in substoichiometric or stoichiometric amount undergoes an undesired reaction with these impurities during the peptide coupling reaction.

The choice of the appropriate reaction temperature depends on the employed coupling reagent as well as on the stability of the peptide. Preferably, the peptide coupling reaction is carried out at a reaction temperature of from −15° C. to 50° C., more preferably from −10° C. to 30° C., even more preferably from 0° C. to 25° C.

Preferably, the peptide coupling reaction is carried out at the atmospheric pressure. However, it is also possible to carry out the peptide coupling reaction at a pressure which is higher or slightly lower than the atmospheric pressure.

Preferably, the peptide coupling reaction is carried out under an ambient atmosphere. However, an atmosphere of a protective gas such as nitrogen or argon is also preferable.

In the present application, the term “reaction time” refers to the time required until the conversion of the reaction is substantially complete. The conversion of the reaction is considered to be substantially complete, once the amount of the starting material used in substoichiometric or stoichiometric amount decreases to less than 5 mol.-% of its initial amount, preferably to less than 2 mol.-% of its initial amount. The progress of the reaction can be monitored by analytical methods known in the art, for instance, by analytical high-performance liquid chromatography (HPLC), thin layer chromatography (TLC), mass spectrometry (MS) or HPLC-MS, whereby HPLC is particularly preferred for this purpose.

Preferably, the reaction time for the peptide coupling reaction ranges from 15 min to 20 h, more preferably from 30 min to 5 h, even more preferably from 30 min to 2 h.

The term “part” in this description of reaction conditions of the peptide coupling reaction is meant to be a factor of the parts by weight of the total weight of the peptides and/or amino acids employed as starting materials for the peptide coupling reaction. Preferably, from 1 to 30 parts, more preferably from 5 to 10 parts of the reaction solvent are used.

Preferably, from 0.9 to 5 mol equivalents, more preferably from 1 to 1.5 mol equivalents of coupling reagent is used, the mol equivalent being based on the mol of reactive C-terminal carboxylic acid groups.

Preferably, from 0.1 to 5 mol equivalents, more preferably from 0.5 to 1.5 mol equivalents of coupling additive is used, the mol equivalent being based on the mol of coupling reagent.

Preferably, from 1 to 10 mol equivalents, more preferably from 2 to 3 mol equivalents, of tertiary base is used, the mol equivalent being based on the mol of coupling reagent.

Any peptide is obtainable by the process for preparation of a peptide in liquid phase of the present invention.

Preferably, the peptide obtained by the process for preparation of a peptide in liquid phase of the present invention comprises 100 or less amino acid residues, more preferably 50 or less amino acid residues, most preferably 20 or less amino acid residues. The amino acids of the peptide can be D- and L-α-amino acids, n-amino acids as well as other organic compounds containing at least one primary and/or secondary amino group and at least one carboxylic acid group. Preferably, the amino acids of the peptide obtained by the process for preparation of a peptide in liquid phase of the present invention are α-amino acids, even more preferably L-α-amino acids, whereby proteinogenic amino acids are particularly preferred.

Preferably, after the process for extraction the organic layer containing the peptide is partially evaporated. In the present application the obtained layer is thus designated as “partially evaporated organic layer”. The temperature at which the partial evaporation takes place is not particularly limited and depends on the properties of the mixture of the organic solvent 1 with the organic solvent 2 as well as on the thermal stability of the peptide. It is preferred that the partial evaporation of the organic layer is carried out at a temperature of from 30° C. to 50° C. If required, the partial evaporation of the organic layer is carried out under reduced pressure of from 20 mbar to 1000 mbar (20 hPa to 1000 hPa). A person skilled in the art is aware that the pressure at which the partial evaporation of the organic layer takes place is preferably adjusted according to the desired evaporation temperature.

Since the organic solvent 1 and the organic solvent 2 are sufficiently volatile, the partial evaporation of the organic layer containing the peptide can be easily carried out.

In one of the embodiments of the present invention, the organic layer containing the peptide is directly evaporated until dryness and the remaining residue is dissolved in a solvent which is distinct from the organic solvent 1 and the organic solvent 2. However, if the organic layer containing the peptide comprises more than 60 vol.-% of a solvent selected from the group consisting of MeTHF and THF the complete evaporation until dryness is preferably avoided for safety reasons. Instead, the partial evaporation of the organic layer containing the peptide can be carried out, followed by an addition of toluene and a subsequent evaporation until dryness.

Because at least one of the solvents present in the organic layer forms an azeotrope with water, the traces of water in the organic layer containing the peptide are efficiently removed during the process of partial evaporation. The removal of the traces of water during the process of partial evaporation is particularly efficient if the organic solvent 1 is 2-methyltetrahydrofuran.

In one of the preferred embodiments, the substantial part of the peptide is precipitated upon combining the partially evaporated organic layer with an organic solvent 3.

In another preferred embodiment of the present invention, the organic layer containing the peptide is evaporated until dryness and the remaining residue is dissolved in a solvent which is distinct from the organic solvent 1 and the organic solvent 2. The obtained solution is combined with the organic solvent 3, whereby the peptide precipitation takes place.

The volume ratio partially evaporated organic layer:organic solvent 3 employed during the process for precipitation of the peptide has a strong impact on the completeness of the process for precipitation and on the properties of the precipitated peptide. In the following the ratios are given as volume to volume ratios.

It is preferred that the volume ratio partially evaporated organic layer:organic solvent 3 ranges from 1:20 to 1:1. Preferably, this volume ratio ranges from 1:12 to 1:2. It is particularly preferred that this volume ratio ranges from 1:6 to 1:3.

The organic solvent 3 is preferably selected from organic solvents having a boiling point of less than 160° C. at the atmospheric pressure. Preferably, the solubility of the peptide in the organic solvent 3 is lower than in the organic solvent 1 or in the mixture of the organic solvent 1 and the organic solvent 2. The organic solvent 3 is preferably selected from the group consisting of acetonitrile, diethyl ether, diisopropyl ether, n-heptane and toluene, more preferred from the group consisting of acetonitrile, diethyl ether, diisopropyl ether and toluene and particularly preferred from the group consisting of diisopropyl ether and toluene. In a most preferred embodiment, the organic solvent 3 is diisopropyl ether.

Because the partially evaporated organic layer containing the peptide is substantially free of the polar aprotic solvent, the amount of the organic solvent 3 required for the precipitation of the peptide is significantly lower than in the precipitation processes of the prior art, which use crude reaction mixtures resulting from the peptide coupling reaction. In addition, contrary to the precipitation processes of the prior art, the precipitated peptide is a non-sticky solid material.

Preferably, during the precipitation process at least 80 wt.-% of the peptide present in the partially evaporated organic layer precipitates as a solid material. It is even more preferred that at least 90 wt.-% of the peptide present in the partially evaporated organic layer precipitates as a solid material. It is yet even more preferred that at least 95 wt.-% of the peptide present in the partially evaporated organic layer precipitates as a solid material. It is particularly preferred that at least 98 wt.-% of the peptide present in the partially evaporated organic layer precipitates as a solid material.

The temperature at which the precipitation process is carried out (this temperature is hereinafter designated as precipitation temperature) depends on the composition of the partially evaporated organic layer, choice of the organic solvent 3 and on the properties of the peptide.

The precipitation temperature has a strong influence on the completeness of the precipitation of the peptide and on the physical properties of the precipitated peptide. Preferably, the precipitation process is carried out at the precipitation temperature of from −10° C. to 60° C., whereby the precipitation temperature of from −10° C. to 30° C. is even more preferred. It is, however, particularly preferred that the precipitation temperature ranges from −10° C. to 0° C.

Since the partially evaporated organic layer containing the peptide is substantially free of the polar aprotic solvent, the precipitated peptide can be easily separated by filtration. Therefore, the time required for the filtration process is significantly shortened. Preferably, the precipitated peptide is separated by filtration and dried under reduced pressure.

It is also possible, however, to separate the precipitated peptide by centrifugation.

If desired, the filtrate collected during the filtration can be subjected again to a partial evaporation and to a subsequent precipitation, so that a second batch of the precipitated peptide can be collected.

In another embodiment of the present invention, the partially evaporated organic layer containing the peptide is directly treated with a reagent cleaving one or several PGs of the peptide. Because the partially evaporated organic layer containing the peptide is substantially free of the polar aprotic solvent, the choice of the reagents for the cleavage of one or several PGs of the peptide is not particularly limited. For instance, the partially evaporated organic layer containing the peptide can be treated with an acidolytic reagent, whereby no undesired reactions between the acidolytic reagent and polar aprotic solvent or inhibition of the cleavage take place. This embodiment of the present invention is particularly preferable if the N-terminal PG of the peptide is tert-butoxycarbonyl (Boc) group.

In other embodiments of the present invention, the partially evaporated organic layer is used for carrying out other reactions such as disulphide bridge formation.

In another embodiment of the present invention, the reagent cleaving one or several PGs of the peptide is added directly to the reaction mixture resulting from a peptide coupling reaction. After the cleavage of the targeted PG is complete, the resulting peptide is extracted from the reaction mixture. This embodiment of the present invention is particularly suitable if the N-terminal PG of the peptide is fluorenyl-9-methoxycarbonyl (Fmoc) group.

In one particular embodiment the peptide after PG cleavage is extracted in MeTHF or using a mixture of MeTHF with an organic solvent 2. This is typically the case with Fmoc protected peptides that are difficult to keep in solution without NMP or DMF. After Fmoc cleavage these can be extracted in an organic layer containing MeTHF.

With Boc protected peptides, it is the opposite, NMP and DMF have to be removed for Boc cleavage, but these peptides are usually soluble in the presence of TFA>5 vol-% in toluene, ethylacetate or eventually heptanes.

In yet another embodiment of the present invention, the organic layer containing the peptide is evaporated until dryness as described above, the remaining residue is dissolved in a solvent distinct from the organic solvent 1 and the organic solvent 2 and the reagent cleaving one or several PGs of the peptide is added thereto afterwards.

### Protecting Groups

Protecting groups (PGs), be it for protecting functional groups in side chains of amino acids or peptides or for the protection of N-terminal amino groups or C-terminal carboxylic acid groups of amino acids or peptides, are for the purpose of the present invention classified into four different groups:

1. PGs cleavable under basic cleaving conditions, in the following called “basic type PGs”,

2. PGs cleavable under strongly acidic cleaving conditions but not cleavable under mildly acidic cleaving conditions, in the following called “strong type PGs”,

3. PGs cleavable under mildly acidic cleaving conditions, in the following called “weak type PGs”,

4. PGs cleavable under reductive cleaving conditions, in the following called “reductive type PGs”, and

5. PGs cleavable under saponification cleaving conditions, in the following called “saponification type PGs”.

PGs and typical reaction conditions, parameters and reagents for cleaving PGs, which are conventionally used in the process for preparation of a peptide in liquid phase of the present invention, are known in the art, e.g. T. W. Greene, P. G. M. Wuts “Greene's Protective Groups in Organic Synthesis” John Wiley & Sons, Inc., 2006; or P. Lloyd-Williams, F. Albericio, E. Giralt, “Chemical Approaches to the Synthesis of Peptides and Proteins” CRC: Boca Raton, Fla., 1997.

Basic cleaving conditions involve treatment of the peptide with a basic cleaving solution. Preferably, the basic cleaving solution consists of a basic reagent and a solvent. Basic reagents used in the present invention are preferably secondary amines, more preferably the basic reagent is selected from the group consisting of diethylamine (DEA), piperidine, 4-(aminomethyl)piperidine, tris(2-aminoethyl)amine (TAEA), morpholine, dicyclohexylamine, 1,3-cyclohexanebis(methylamine)-piperazine, 1,8-diazabicyclo[5.4.0]undec-7-ene and mixtures thereof. Even more preferably, the basic reagent used in the process for preparation of a peptide in liquid phase of the present invention is selected from the group consisting of DEA, TAEA and piperidine.

The basic cleaving solution can also comprise an additive, preferably selected from the group consisting of 6-chloro-1-hydroxy-benzotriazole, 1-hydroxy-7-azabenzotriazole, 1-hydroxybenzotriazole and ethyl-2-cyano-2-hydroxyiminoacetate and mixtures thereof.

Preferably, the solvent of the basic cleaving solution is identical to the polar aprotic solvent employed for the peptide coupling reaction. Thus, the solvent for the basic cleaving solution is preferably selected from the group consisting of DMF, DMA and NMP. Alternatively, the peptide containing organic layer which is obtained by the process for extraction of a peptide from a reaction mixture resulting from a peptide coupling reaction can be evaporated until dryness as described above. The remaining residue can be dissolved in one of the solvents selected from the group consisting of DMF, DMA, pyridine, NMP, ACN or a mixture thereof and subsequently treated with a basic cleaving solution. DMF or NMP may be necessary to keep the peptide in solution in Fmoc cleavage reaction mixture as shown in example 1.

The terms “part” and “wt.-%” in the description of basic, strongly acidic, mildly acidic and reductive cleaving conditions are meant to be a factor of the parts by weight of the peptide carrying the corresponding groups PG(s) which are being cleaved. For instance, the expression “5 parts of basic cleaving solution are used” means that 5 g of basic cleaving solution are used for the treatment of each 1 g of the peptide carrying a basic type PG.

Preferably, from 5 to 20 parts, more preferably from 5 to 15 parts of basic cleaving solution are used. Preferably, the amount of basic reagent ranges from 1 to 30 wt.-%, more preferably from 10 to 25 wt.-%, even more preferably from 15 to 20 wt.-%, with the wt.-% being based on the total weight of the basic cleaving solution.

Strongly acidic cleaving conditions, as defined in the present invention, involve treatment of the peptide with a strongly acidic cleaving solution. The strongly acidic cleaving solution comprises an acidolytic reagent. Acidolytic reagents are preferably selected from the group consisting of Brønsted acids, such as TFA, hydrochloric acid (HCl), aqueous hydrochloric acid (HCl), liquid hydrofluoric acid (HF) or trifluoromethanesulfonic acid, Lewis acids, such as trifluoroborate diethyl ether adduct or trimethylsilylbromid, and mixtures thereof.

The strongly acidic cleaving solution preferably comprises one or more scavengers, selected from the group consisting of dithiothreitol, ethanedithiol, dimethylsulfide, triisopropylsilane, triethylsilane, 1,3-dimethoxybenzene, phenol, anisole, p-cresol and mixtures thereof. The strongly acidic cleaving solution can also comprise water, a solvent or a mixture thereof, the solvent being stable under strong cleaving conditions. Preferably, the solvent of the strongly acidic cleaving solution is identical to the solvent present in the partially evaporated organic layer containing the peptide. Thus, the solvent for the strongly acidic cleaving solution is a combination of the organic solvent 1 and the organic solvent 2. Alternatively, the organic layer containing the peptide can be evaporated until dryness as described above and the remaining residue can be dissolved in one of the solvents selected from the group consisting of ACN, toluene, DCM, TFA, and mixtures thereof. Because the organic solvent 1 and the organic solvent 2 are sufficiently volatile, the evaporation of the organic layer can be easily carried out.

Preferably, from 10 to 30 parts, more preferably from 15 to 25 parts, even more preferably from 19 to 21 parts of strongly acidic cleaving solution are used. Preferably, the amount of acidolytic reagent ranges from 30 to 350 wt.-%, more preferably from 50 to 300 wt.-%, even more preferably from 70 to 250 wt.-%, especially from 100 to 200 wt.-%, with the wt.-% being based on the total weight of the strongly acidic cleaving solution. Preferably, from 1 to 25 wt.-% of total amount of scavenger is used, more preferably from 5 to 15 wt.-%, with the wt.-% being based on the total weight of the strongly acidic cleaving solution.

Mildly acidic cleaving conditions according to the present invention involve treatment of the peptide with a weakly acidic cleaving solution. The weakly acidic cleaving solution comprises an acidolytic reagent. The acidolytic reagent is preferably selected from the group consisting of Brønsted acids, such as TFA, trifluoroethanol, hydrochloric acid (HCl), acetic acid (AcOH), mixtures thereof and/or with water.

The weakly acidic cleaving solution can also comprise water, a solvent or a mixture thereof, the solvent being stable under weak cleaving conditions. Preferably, the solvent of the weakly acidic cleaving solution is identical to the solvent present in the partially evaporated organic layer containing the peptide. Thus, the solvent for the weakly acidic cleaving solution is a combination of the organic solvent 1 and the organic solvent 2. Alternatively, the organic layer containing the peptide can be evaporated until dryness as described above and the remaining residue can be dissolved in one of the solvents selected from the group consisting of ACN, toluene, DCM, TFA, and mixtures thereof.

Preferably, from 4 to 20 parts, more preferably from 5 to 10 parts, of weakly acidic cleaving solution are used. Preferably, the amount of acidolytic reagent ranges from 0.01 to 5 wt.-%, more preferably from 0.1 to 5 wt.-%, even more preferably from 0.15 to 3 wt.-%, with the wt.-% being based on the total weight of the weakly acidic cleaving solution.

Reductive cleaving conditions employed in one of the embodiments of the present invention involve treatment of the peptide with a reductive cleaving mixture. The reductive cleaving mixture comprises a catalyst, a reducing agent and a solvent.

The catalysts employed for the reductive cleaving conditions are selected from the group consisting of derivatives of Pd(0), derivates of Pd(II) and catalysts containing metallic palladium, more preferably selected from the group consisting of Pd[PPh₃]₄, PdCl₂[PPh₃]₂, Pd(OAc)₂ and palladium on carbon (Pd/C). Pd/C is particularly preferred.

The reducing agent is preferably selected from the group consisting of Bu₄N⁺BH₄, NH₃BH₃, Me₂NHBH₃, tBu-NH₂BH₃, Me₃NBH₃, HCOOH/DIPEA, sulfinic acids comprising PhSO₂H, tolSO₂Na and i-BuSO₂Na and mixtures thereof as well as molecular hydrogen; more preferably the reducing agent is tolSO₂Na or molecular hydrogen.

Preferably, the solvent employed under reductive cleaving conditions is identical to the solvent present in the partially evaporated organic layer containing the peptide i.e. is a combination of the organic solvent 1 and the organic solvent 2. Alternatively, the organic layer containing the peptide can be evaporated until dryness as described above and the remaining residue can be dissolved in one of the solvents selected from the group consisting of NMP, DMF, DMA, pyridine, ACN and mixtures thereof; more preferably the solvent is NMP, DMF or a mixture thereof. Preferably, the peptide is soluble and dissolved in the solvent employed under reductive cleaving conditions.

Preferably, from 4 to 20 parts, more preferably from 5 to 10 parts, of reductive cleaving solution are used.

Saponification cleaving conditions involve treatment of the peptide with a saponification cleaving solution. Preferably, the saponification cleaving solution consists of a saponification reagent and a solvent. Saponification reagents used in the present invention are preferably hydroxides of alkaline and earth alkaline metals, more preferably the saponification reagent is selected from the group consisting of sodium hydroxide, lithium hydroxide and potassium hydroxide. Even more preferably, the saponification reagent used in the process for preparation of a peptide in liquid phase of the present invention is sodium hydroxide.

Preferably, the solvent of the saponification cleaving solution comprises a mixture of water with a solvent selected from the group consisting of THF, MeTHF, ethanol, methanol and dioxane.

According to the present invention, the basic type PGs are not cleavable under strongly acidic or mildly acidic cleaving conditions. Preferably, the basic type PGs are not cleavable under strongly acidic, weak or reductive cleaving conditions.

Under the term “strong type PGs” are protecting groups understood which are not cleavable under mildly acidic or basic cleaving conditions. Preferably, the strong type PGs are not cleavable under mildly acidic, basic or reductive cleaving conditions. Usually strong acidic PGs like Bzl are cleaved by hydrogenation. Typically, the global deprotection of a peptide is carried out by hydrogenation under very mild conditions.

The weak type PGs are not cleavable under basic cleaving conditions, but they are cleavable under strongly acidic cleaving conditions. Preferably, the weak type PGs are not cleavable under basic or reductive cleaving conditions, but they are cleavable under strongly acidic cleaving conditions.

According to one of the embodiments of the present invention, the basic type PG is preferably Fmoc. Preferably, the strong type PGs are selected from the group consisting of Boc, tBu, OtBu and Cbz. Preferably, the weak type PGs are selected from the group consisting of Trt and 2-chlorophenyldiphenylmethyl group. Preferably, the reductive type PGs are selected from the group consisting of Bzl, N-methyl-9H-xanthen-9-amino group and Cbz. Preferably, the saponification type PG is OMe.

In the process for preparation of a peptide in liquid phase of the present invention, the N-terminal PG of the peptide is removed in a deprotection reaction before the subsequent peptide coupling reaction is carried out. According to the present invention, the N-terminal PGs are preferably Fmoc, and Boc.

In one of the embodiments of the present invention, Fmoc is highly preferred for the LPPS as an N-terminal PG because it can be easily removed under basic conditions. Furthermore, the Fmoc as a PG of the N-terminus of the peptide is compatible with the side chain PGs in order to represent an orthogonal system. The term “orthogonal system” is defined in G. Baranay and R. B. Merrifield (JACS, 1977, 99, 22, pp. 7363-7365).

In yet another embodiment of the present invention, Boc is highly preferred as an N-terminal PG of the peptide for process for the preparation of a peptide in liquid phase. Its removal can be carried out under strongly acidic conditions. Usage of Boc PG of the N-terminus is also compatible with the side chain PGs in order to represent an orthogonal system.

According to the present invention, the C-terminal PG of the peptide is removed in the final deprotection step.

Preferred C-terminal PGs are OtBu, Blz, OMe, NH₂, as well as 2-chlorophenyl-diphenylmethylester or N-methyl-9H-xanthen-9-amide.

In one of the embodiments of the present invention, Bzl is highly preferred for the process for preparation of a peptide in liquid phase as a C-terminal PG because it can be easily removed under reductive cleaving conditions described above. Furthermore, the Bzl PGs of the C-terminus is compatible with the side chain PGs in order to represent an orthogonal system.

In another embodiment of the present invention, OtBu as a C-terminal PG is used for the process for preparation of a peptide in liquid phase. Its removal can be carried out under strongly acidic cleaving conditions as described above. Usage of OtBu PG of the C-terminus is also compatible with the side chain PGs in order to represent an orthogonal system.

In another embodiment of the present invention, OMe as a C-terminal PG is used for the process for preparation of a peptide in liquid phase. OMe can be easily cleaved by saponification and is particularly useful if the N-terminal PG of the peptide is Boc.

In yet another embodiment of the present invention, the solubility of the peptide in the organic layer can be additionally increased by using a hydrophobic PG for the C-terminus of the peptide. For this purpose, the C-terminal carboxylic acid group of the peptide can be protected with a weak type PGs, which are cleavable in mildly acidic conditions, such as a 2-chlorophenyldiphenylmethylester or N-methyl-9H-xanthen-9-amide. These PGs are particularly useful for the synthesis of peptide fragments, which, in turn can be employed in a convergent peptide synthesis. These C-terminal carboxylic acid protecting groups have another important advantage: they are cleaved in mildly acidic conditions, allowing for the liquid phase synthesis of protected peptides, as an alternative to SPPS, that are used as peptide fragments in a convergent synthesis strategy. Actually, 2-chlorophenyldiphenylmethylester and N-methyl-9H-xanthen-9-amide are chemical functions that are used as linkers on SPPS resins for the synthesis of protected peptide fragments.

According to the present invention, it is desirable that the hydroxy-, amino-, thio- and carboxylic acid groups of the amino acids side chains of the peptide obtained by the process for preparation of a peptide in liquid phase are protected with suitable PGs, so that undesired side reactions are avoided. In addition, usage of the side chain PGs generally improves the solubility of the peptide in the polar aprotic solvents as well as in the organic solvent 1 or/and in the combination of the organic solvent 1 and the organic solvent 2.

Generally, side chain PGs are chosen in such a way that they are not removed during the deprotection of the N-terminal amino groups during the process for preparation of a peptide in liquid phase. Therefore, the PG of the N-terminal amino groups or C-terminal carboxylic acid groups and any side chain PG are typically different, preferably they represent an orthogonal system.

According to the present invention, the preferred side chain groups are tBu, Trt, Boc, OtBu and Cbz.

Once the amino acid sequence of the peptide obtained by the process for preparation of a peptide in liquid phase is identical to the amino acid sequence of the target peptide, preferably the N-terminal PG, the C-terminal PG and any side chain PG are removed so that the unprotected target peptide is obtained. This step is called global deprotection. Preferably, the PGs used during the process for preparation of a peptide in liquid phase are selected to allow global deprotection under mildly acidic, strongly acidic or reductive cleaving conditions, as defined above, depending on the nature of PGs.

Any side chain PGs are typically retained until the end of the LPPS. Global deprotection can be carried out under conditions applicable to the various side chain PGs, which have been used. In the case that different types of the side chain PGs are chosen, they may be cleaved successively e.g. this is the case for the synthesis of a branched peptide. Advantageously, the side chain PGs are chosen in such a way so that they are cleavable simultaneously and more advantageously concomitantly with N-terminal PG or with C-terminal PG of the peptide prepared by LPPS.

In one of the embodiments of the present invention it is possible that the N-terminal PG of the peptide in the partially evaporated organic layer is directly removed. Thus, in this case, the precipitation of the peptide upon usage of the organic solvent 3 is not required and LPPS of the present invention can be carried out without an isolation of the intermediate peptides, e.g. as a continuous LPPS.

Depending on the nature of the N-terminal PG of the peptide, appropriate cleaving conditions can be chosen for this step.

If the N-terminal PG of the peptide is a strong type PG or a weak type PG, as defined above, the organic layer containing the peptide is preferably treated with TFA or HCl. Because the organic layer containing the peptide is substantially free from the polar aprotic solvents, the removal of the N-terminal PG of the peptide is not inhibited by an undesired reaction between TFA or HCl and the polar aprotic solvent. In one of the embodiments of the present invention, the N-terminal PG of the peptide is Boc group.

If the N-terminal PG of the peptide is a basic type PG, as defined above, the peptide can be deprotected upon usage of an organic base, as known in the prior art. Preferably, for this purpose the reaction mixture resulting from a peptide coupling reaction is directly treated with a basic reagent selected from the group consisting of DEA, TAEA and piperidine and the peptide with an unprotected N-terminus is extracted from this reaction mixture. Alternatively, the organic layer containing the peptide is treated with the basic reagent. Alternatively, the organic layer containing the peptide can be evaporated until dryness as described above and the remaining residue can be dissolved in one of the solvents selected from the group consisting of DMF, DMA, pyridine, NMP or a mixture thereof and subsequently treated with the basic reagent.

In one of the preferred embodiments of the present invention, the N-terminal PG of the peptide is fluorenyl-9-methoxycarbonyl (Fmoc) group. Cleavage of the Fmoc group of the peptide is accompanied by formation of dibenzofulvene. If DEA or piperidine is used as a basic reagent and the solvent of the basic cleaving solution is acetonitrile, the resulting solution containing the peptide with an unprotected N-terminus is subsequently washed with a hydrocarbon such as e.g. n-heptane so that dibenzofulvene is substantially removed. If TAEA is used as a basic reagent for the cleavage of the Fmoc group, the resulting solution is subsequently subjected to the extraction process of the present invention. Thus, the solution containing the peptide with an unprotected N-terminus is substantially free of dibenzofulvene before a subsequent peptide coupling reaction is carried out.

After the cleavage of the N-terminal PG of the peptide, the solution containing the peptide with an unprotected N-terminus can be at least partially evaporated and employed for the subsequent peptide coupling reaction or, alternatively, to the global deprotection step.

Thus, the present invention provides continuous LPPS methodology, which has a number of advantages over commonly used SPPS methodology.

Concentrations of reagents present in the reaction mixture during the peptide coupling reactions and deprotection reactions in the case of the continuous LPPS of the present invention are higher than in the case of SPPS. As a consequence, the corresponding reaction times are shorter and batch reactors with a lower capacity can be used for the synthesis of a given amount of target peptide. The total time required for the synthesis of a peptide carried out by the continuous LPPS of the present invention is nearly the same as the total time required for its synthesis if SPPS is used. Thus, use of the continuous LPPS of the present invention leads to reduced operating costs.

A peptide coupling reaction in the LPPS of the present invention requires a lower excess of an amino acid or a peptide having an unprotected C-terminal carboxylic acid group (1.1-1.2 equivalents) than the corresponding peptide coupling reaction in SPPS (1.5 equivalents or more). Moreover, SPPS further requires a high amount of solvents for rinsing the resin after each peptide coupling step. Thus, the amount of solvents required in the case of SPPS is significantly higher than in the case of the continuous LPPS of the present invention. Hence, use of continuous LPPS of the present invention leads to a significant reduction of material costs in comparison to use of SPPS.

In addition thereto, the scaling up of the continuous LPPS process of the present invention is known to be easier than the scaling up of the corresponding SPPS process, and the target peptide prepared by the continuous LPPS of the present invention has a higher purity than the corresponding peptide prepared by SPPS.

In summary, the continuous LPPS of the present invention provides a number of advantages over other methodologies for peptide synthesis, known in the prior art, and is particularly useful for the preparation of peptides on an industrial scale.

### DESCRIPTION OF THE DRAWINGS

FIG. 1 shows a contour plot illustrating the NMP content (g/L) in the organic layer of the ternary mixture NMP/MeTHF/water (black circles represent the compositions of the experimental mixtures, those prepared in duplicates are labelled with “2 x”).

FIG. 2 shows a contour plot illustrating the volume of the organic layer (mL) of the ternary mixture NMP/MeTHF/water (black circles represent the compositions of the experimental mixtures, those prepared in duplicates are labelled with “2 x”).

FIG. 3 shows a contour plot illustrating the NMP content (g/L) in the organic layer of the ternary mixture NMP/MeTHF/NaCl solution (black circles represent the compositions of the experimental mixtures, those prepared in duplicates are labelled with “2 x”).

FIG. 4 shows a contour plot illustrating the volume of the organic layer (mL) of the ternary mixture NMP/MeTHF/NaCl solution (black circles represent the compositions of the experimental mixtures, those prepared in duplicates are labelled with “2 x”).

FIG. 5 shows a calculated contour plot of the extraction yield of the pentapeptide H-Leu-Trp(Boc)-Val-Asn(Trt)-Ser(tBu)-NH₂ in water as a function of the relative composition of the system MeTHF/NMP/water (black circles represent the compositions of the experimental mixtures, those prepared in triplicate are labelled with “3 x”).

FIG. 6 shows a diagram representing the dependency of concentration of NMP in organic layer as a function of the composition of the system NMP/MeTHF/THF/water.

FIG. 7 illustrates the influence of residual DMF on the rate of removal of the Boc protecting group of peptide Boc-Pro-Ile-Leu-Pro-Pro-Glu(OBzl)-Glu(OBzl)-Tyr(Bzl)-Leu-OBzl.

Test #1: Boc-Pro-Ile-Leu-Pro-Pro-Glu(OBzl)-Glu(OBzl)-Tyr(Bzl)-Leu-OBzl was isolated using extraction with DCM.
Test #3: Boc-Pro-Ile-Leu-Pro-Pro-Glu(OBzl)-Glu(OBzl)-Tyr(Bzl)-Leu-OBzl was isolated using extraction with EtOAc.
Test #5: Boc-Pro-Ile-Leu-Pro-Pro-Glu(OBzl)-Glu(OBzl)-Tyr(Bzl)-Leu-OBzl was isolated using extraction with MeTHF.

FIG. 8 shows an image of the peptide Boc-Ser(Bzl)-Phe-Pro-Ile-Leu-Pro-Pro-Glu(OBzl)-Glu(OBzl)-Tyr(Bzl)-Leu(OBzl), which was isolated according to the process of the present invention.

### EXAMPLES

The following non-limiting examples will illustrate representative embodiments of the invention in detail.

All experiments were carried out at room temperature of 20±3° C. and atmospheric pressure of 1013±50 kPa if not specified otherwise.

### Methods Description

### A) HPLC Analysis

Detection in HPLC method A was done with a UV photodiode array detector.

### Step 1 Sample Preparation:

Mobile Phase A: 0.1 Vol.-% TFA in water

Mobile Phase B: 0.085 Vol.-% TFA in ACN

### Step 2 Chromatography Conditions:

Method MIH-009-3TG9
Column: Phenomenex Luna C8(2) 5 μm 250×4.6 mm
Oven temperature: 40° C.
Flow rate: 1.50 mL/min
Detector wavelength: 215 nm
Gradient run time: 30 min
Gradient composition: 22 to 52% B in 15 min, 52 to 82% B in 5 min, 82 to 98% B in 5 min, 98% B in 5 min

Method MIH-009-2TG11
Column: Purospher Star RP18 55×4 mm
Oven temperature: 40° C.
Flow rate: 2.0 mL/min
Detector wavelength: 215 nm
Gradient run time: 15 min
Gradient composition: 2 to 78% B in 5 min, 78 to 98% B in 10 min

Method MIH-009-RTTG1
Column: Purospher Star RP18 55×4 mm
Oven temperature: 40° C.
Flow rate: 2.0 mL/min
Detector wavelength: 215 nm
Gradient run time: 15 min
Gradient composition: 2 to 98% B in 5 min, 98% B in 5 min

Method MIH-009-025TG3
Column: XBridgeC18 5μ 150×4.6 mm
Oven temperature: 40° C.
Flow rate: 1.5 mL/min
Detector wavelength: 215 nm
Gradient run time: 20 min
Gradient composition: 2 to 98% B in 15 min, 98% B in 5 min

Method MIH-009-397TG15
Column: Vydac 214TP5415 C4 250×4.6 mm
Oven temperature: 40° C.
Flow rate: 1.5 mL/min
Detector wavelength: 215 nm
Gradient run time: 2 min
Gradient composition: 33 to 78% B in 25 min

### Step 3 Chromatographic Profile Analysis:

The composition of the isolated products was determined by the measurement of the areas of all chromatography peaks. The determined purity of the expected products corresponds to the area-% of the corresponding product peaks.

### 1. Apparatus and Equipment

Gas chromatograph: GC equipped with a flame ionization detector and an automatic injector system coupled with acquisition software
Analytical GC column: Fused silica column, length 50 m; 0.53 mm internal diameter; stationary phase: CP SIL 8CB DF=5.0 μm
Reagents: Methanol (analytical grade)

### 2. Sample Preparation

### Test and Reference Solution

In a 10 mL volumetric flask, add accurately 400 μL of sample and make up to volume with methanol.

### 3. Chromatographic Conditions

Carrier Gas: Helium 30 kPa
Oven temperature: 35° C., 14 minutes 5° C./min 55° C., 3 minutes 5° C./min 110° C., 5 minutes 10° C./min 225° C., 5 minutes
Injector temperature: 225° C.
Detector temperature: 260° C.
Injected volume: 1 μL
Injection mode: Split
Split flow: 85 mL/min
Ratio: 24

### Filterability Measurements

The mixtures containing precipitated peptides were transferred into a 2.7 cm diameter filtration column equipped with a 20 μm pore size filter. Filtrations were carried out at 20° C. under a pressure of 50 mbar. The flow rate and the cake heights were measured and the filterability coefficient K was calculated as:

K=volume of mother liquor(mL)×cake heights(cm)/filter surface(cm²)/pressure (bar)/filtration time(min).

### Design of Experiments

The Design of experiments (DOE) was performed upon using the DOE software package Design-Expert® 8 of Stat-Ease, Inc.

### a) Extraction of NMP in the Systems MeTHF/Water and MeTHF/NaCl Solution

The present example demonstrates that the volume of the organic layer and its NMP content is dependent on the composition of the biphasic systems NMP/MeTHF/water and NMP/MeTHF/NaCl solution. This dependency was verified with designed biphasic systems in which the volume fractions of NMP and MeTHF as well as the NaCl content in water were systematically varied in a quadratic design mode while keeping the overall volume constant. In order to investigate the influence of the NaCl content in water, the same set of biphasic systems was prepared with pure water (see Table 1a) and with a 150 g/L NaCl solution (see Table 1b). After stirring these biphasic systems, they were left for completion of the phase separation in a metering vessel and the volume of the organic layer was measured. The NMP content of the organic layer was measured by gas chromatography. Statistical models were developed to determine the volume of the organic layer and its NMP content as mathematical functions of the volume fractions of NMP, MeTHF and water in the overall biphasic system composition (these volume fractions summing to 1).

a) In the absence of NaCl, the NMP content Ln (g/L) in the organic layer is given by the quadratic mixture model (with R²=0.954):

Ln(NMP in organic layer)=5.7*MeTHF volume fraction−17.1*NMP volume fraction−6.9*H₂O volume fraction+102.8*NMP volume fraction*H₂O volume fraction

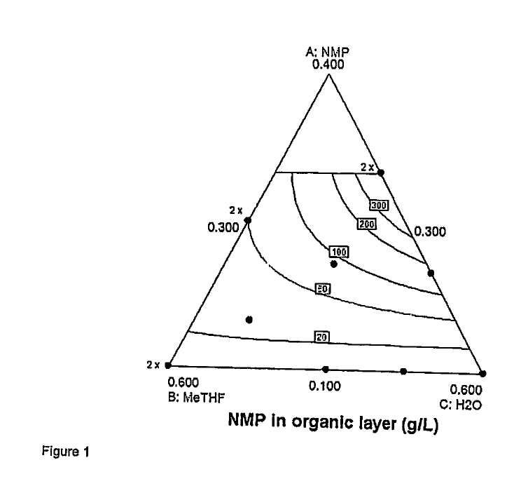

This model of the ternary mixture NMP/MeTHF/water is graphically represented as a contour plot depicted in FIG. 1.

b) The volume of organic layer in the absence of NaCl is given by the linear mixture model (with R²=0.992):

Vol. organic layer(mL)=22.6*MeTHF volume fraction−10.4*NMP volume fraction−4.7*H₂O volume fraction

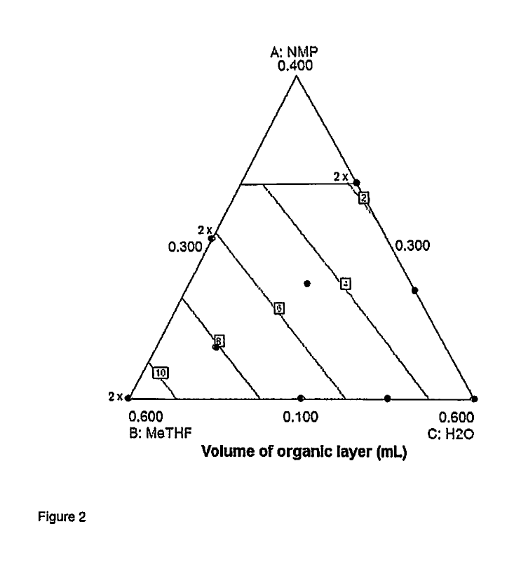

This model of the ternary mixture NMP/MeTHF/water is graphically represented as a contour plot depicted in FIG. 2.

TABLE 1a
Extraction of NMP in the biphasic system NMP/MeTHF/water in the absence
of NaCl.

| | | | | | | | Fraction of |
|---|---|---|---|---|---|---|---|
| | | | | | | NMP | NMP in |
| | Vol NMP | Vol MeTHF | Vol H₂O | Vol aq I | Vol org I | in org I | aqueous |
| Mixture # | (mL) | (mL) | (mL) | (mL) | (mL) | (mg/mL) | layer |
| 1 | 2 | 9.0 | 9.0 | 13 | 7 | 12.8 | 0.95 |
| 2 | 5.0 | 9.0 | 6.0 | 14 | 6.5 | 52.9 | 0.93 |
| 3 | 5.0 | 9.0 | 6.0 | 14 | 6.5 | 66.2 | 0.91 |
| 4 | 3.0 | 10.0 | 7.1 | 12 | 8 | 26.0 | 0.93 |
| 5 | 2.0 | 12.0 | 6.0 | 9 | 11 | 17.1 | 0.90 |
| 6 | 4.1 | 7.8 | 8.1 | 15 | 5 | 37.7 | 0.95 |
| 7 | 6.0 | 6.0 | 8.0 | 19 | 1 | 460.0 | 0.92 |
| 8 | 4.0 | 6.0 | 10.0 | 18 | 2.5 | 282.4 | 0.81 |
| 9 | 2.0 | 7.5 | 10.5 | 15 | 5 | 8.0 | 0.98 |
| 10 | 2.0 | 12.0 | 6.0 | 9 | 11 | 13.0 | 0.92 |
| 11 | 2.0 | 6.0 | 12.0 | 17 | 3 | 7.0 | 0.99 |
| 12 | 6.0 | 6.0 | 8.0 | 18 | 2 | 443.2 | 0.84 |

c) In the presence of aqueous solution containing 150 g/L NaCl, the NMP content Ln′ (g/L) in the organic layer is given by the linear mixture model (with R²=0.958):

Ln′(NMP in organic layer)=25.1*MeTHF volume fraction+297.3*NMP volume fraction−43.0*NaCl solution volume fraction

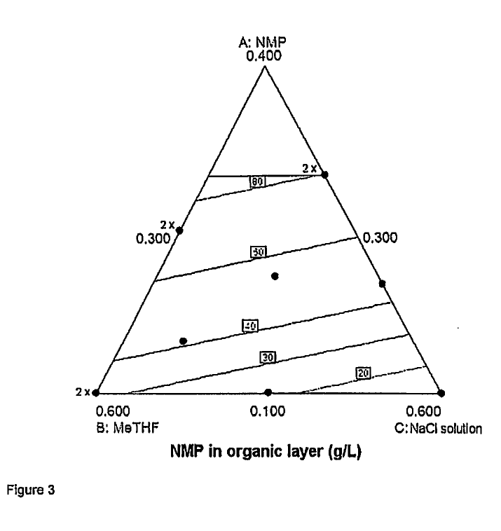

This model of the ternary mixture NMP/MeTHF/NaCl solution is graphically represented as a contour plot depicted in FIG. 3.

d) The volume of the organic layer in the presence of aqueous solution containing 150 g/L NaCl is given by the linear mixture model (with R²=0.991):

Vol. organic layer=20.5*MeTHF volume fraction−1.05*NMP volume fraction−1.08*NaCl solution volume fraction

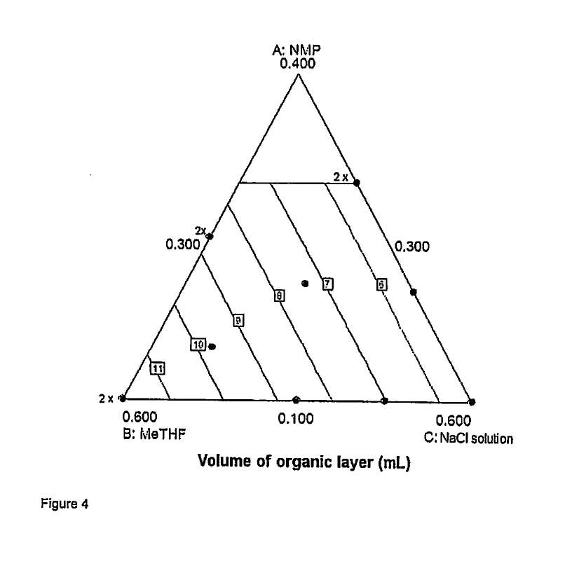

This model of the ternary mixture NMP/MeTHF/NaCl solution is graphically represented as a contour plot depicted in FIG. 4.

TABLE 1b
Extraction of NMP in the biphasic system NMP/MeTHF/NaCl solution
(150 g/L NaCl).

| | | | | | | | Fraction of |
|---|---|---|---|---|---|---|---|
| | | | | | | NMP | NMP in |
| | Vol NMP | Vol MeTHF | Vol H₂O | Vol aq I | Vol org I | in org I | aqueous |
| Mixture # | (mL) | (mL) | (mL) | (mL) | (mL) | (mg/mL) | layer |
| 1 | 2.0 | 9.0 | 9.0 | 12 | 8.5 | 21.1 | 0.91 |
| 2 | 5.0 | 9.0 | 6.0 | 12 | 8.5 | 81.1 | 0.85 |
| 3 | 5.0 | 9.0 | 6.0 | 12 | 8.5 | 74.0 | 0.87 |
| 4 | 3.0 | 10.0 | 7.1 | 11 | 9.5 | 47.4 | 0.84 |
| 5 | 2.0 | 12.0 | 6.0 | 8 | 12 | 28.7 | 0.82 |
| 6 | 4.1 | 7.8 | 8.1 | 13 | 7.5 | 49.7 | 0.91 |
| 7 | 6.0 | 6.0 | 8.0 | 15 | 5.5 | 82.3 | 0.92 |
| 8 | 4.0 | 6.0 | 10.0 | 15 | 5.5 | 42.2 | 0.94 |
| 9 | 2.0 | 7.5 | 10.5 | 13 | 7.5 | 17.2 | 0.93 |
| 10 | 2.0 | 12.0 | 6.0 | 8 | 12 | 27.7 | 0.83 |
| 11 | 2.0 | 6.0 | 12.0 | 15 | 5 | 16.0 | 0.96 |
| 12 | 6.0 | 6.0 | 8.0 | 15 | 5.5 | 71.8 | 0.93 |

In order to achieve an efficient removal of NMP from the organic layer containing the peptide, it is desirable that the NMP content in the organic layer is sufficiently low, preferably below 50 g/L and even more preferred below 20 g/L. Such conditions are found in the lower parts of the ternary mixture diagrams shown in FIGS. 1-4. If NaCl is absent, the lowest NMP content in the organic layer can be obtained at a low NMP volume fraction. On the other hand, a lower MeTHF volume fraction leads to a lower organic layer volume. Therefore, unless the peptide of interest is highly soluble in MeTHF, only the conditions corresponding to the bottom left corner of the ternary diagram are applicable for the process of extraction of the peptide.

In the presence of NaCl, the miscibility of MeTHF and water significantly decreases, so that the volume of the organic layer is larger. Moreover, the presence of NaCl increases the density of the aqueous layer, so that the phase separation is quicker.

Therefore, it can be concluded that it is generally preferred to carry out the process for extraction of a peptide in the presence of NaCl and to repeat the extraction with fresh NaCl solution to reach very low NMP content in the MeTHF layer.

b) Extraction of H-Leu-Trp(Boc)-Val-Asn(Trt)-Ser(tBu)-NH₂ in the System NMP/MeTHF/NaCl Solution

A central composite DoE was performed for the process of extraction of the pentapeptide H-Leu-Trp(Boc)-Val-Asn(Trt)-Ser(tBu)-NH₂. The extraction yield of this peptide was measured from a solution in NMP having a concentration of 200 mg/mL. The relative volumes of MeTHF and of water, as well as the NaCl content in water, were systematically varied. One experiment was carried out for each boundary condition and 3 experiments were carried out for the centre point. The obtained results are shown in Table 2 below.

TABLE 2
Extraction yields of H-Leu-Trp(Boc)-Val-Asn(Trt)-Ser(tBu)—NH₂ as
a function of the composition of the organic and of the aqueous layers.

| | MeTHF | H₂O | NaCl in | Extraction |
|---|---|---|---|---|
| Mixture # | Vx | Vx | water (g/L) | yield (%) |
| 1 | 2 | 2 | 0 | 90.7 |
| 2 | 3.5 | 3.5 | 100 | 100.0 |
| 3 | 3.5 | 5 | 100 | 100.0 |
| 4 | 5 | 3.5 | 100 | 99.4 |
| 5 | 2 | 3.5 | 100 | 99.7 |
| 6 | 3.5 | 2 | 100 | 96.7 |
| 7 | 5 | 2 | 0 | 57.8 |
| 8 | 5 | 5 | 200 | 100.0 |
| 9 | 3.5 | 3.5 | 100 | 100.0 |
| 10 | 3.5 | 3.5 | 100 | 100.0 |
| 11 | 2 | 2 | 200 | 98.1 |
| 12 | 5 | 5 | 0 | 100.0 |
| 13 | 3.5 | 3.5 | 0 | 97.9 |
| 14 | 5 | 2 | 200 | 98.9 |
| 15 | 2 | 5 | 0 | 99.8 |
| 16 | 2 | 5 | 200 | 100.0 |
| 17 | 3.5 | 3.5 | 200 | 100.0 |

MeTHF Vx stands for the volume ratio MeTHF: reaction mixture (RM).
H₂O Vx stands for the volume ratio H₂O: reaction mixture (RM).

A good mathematical model (R²=0.93) was obtained: the extraction yield of the peptide can be calculated as a function of the volume ratio water:reaction mixture (RM) and of the NaCl content in water as:

Extraction yield(%)=0.77+0.089*(water/RM)+0.00059*NaCl(g/L)−0.00012*(water/RM)*NaCl(g/L)−0.0087(water/RM)²

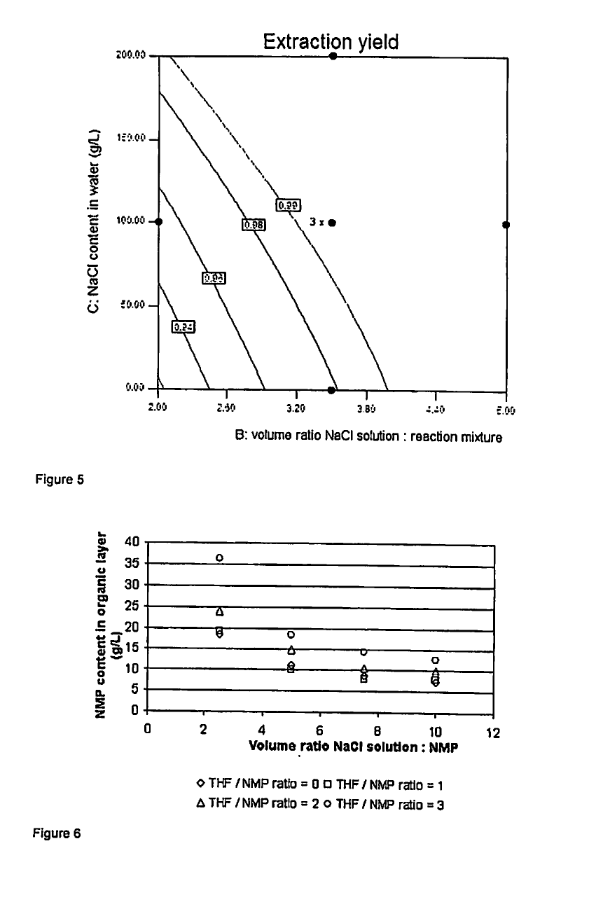

This model can be represented graphically by the contour plot given in FIG. 5. The minimum volume ratio water:reaction mixture needs to sufficiently high in order to reach the extraction yield of over 99%. However, this minimum volume ratio water:reaction mixture is also dependent on the NaCl content in the aqueous layer. Indeed, a higher NaCl content in the aqueous layer leads to a lower miscibility of MeTHF with the aqueous layer. As a consequence, the solubility of the peptide in the aqueous layer is lower. If NaCl is absent in the aqueous layer, the volume ratio water:reaction mixture needs to be higher than 4 in order to reach an extraction yield of over 99%. In the presence of NaCl at 150 g/L in the aqueous layer, a water:reaction mixture ratio=2.7 is sufficient.

It can be considered that the necessary volume ratio water:reaction mixture leading to an extraction yield above 99% is given by:

Water:reaction mixture>4−0.00974*NaCl(g/L)

On the other hand, the volume ratio MeTHF:reaction mixture=2 is always sufficient for an extraction yield to be over 99%.

c) Extraction of NMP in the System MeTHF/THF/NaCl Solution

The following example relates to mixtures consisting of NMP, MeTHF, THF and an aqueous solution containing 150 g/L NaCl. In particular, the dependency between the NMP content (g/L) in the organic layer after the phase separation and the composition of the mixture was investigated. No peptides were present in the systems of the present example.

The volume ratio MeTHF:NMP was 3, whereby the volume ratio NaCl solution:NMP was varied from 2 to 10 and the volume ratio THF:NMP was varied from 0 to 3. The objective of these experiments was to illustrate the interactions between these four components, so these experiments were performed with neat solvents. However, it is noteworthy that the presence of a peptide may change the NMP distribution. The obtained results are represented in FIG. 6.

From FIG. 6 it can be recognised that if the volume ratio THF:NMP is below 2, the NMP content in the organic layer ranges from 10 g/L to 20 g/L, even if the volume ratio water:NMP is low. Typically, if the volume ratio NMP:MeTHF:THF:NaCl solution=1:3:2:5, 90% of NMP is located in the aqueous layer. However, if the volume ratio THF:NMP is higher than 2, the extraction yield of NMP is lower.

Nevertheless, already at a volume ratio NMP:MeTHF:THF:NaCl solution=1:3:3:3, a high extraction yield of more than 99% can be achieved for many peptides, whereas 80% of total NMP is removed into the aqueous layer.

d) Extraction of NMP in Systems MeTHF/THF/NaCl Solution, EtOAc/THF/NaCl Solution and Toluene/THF/NaCl Solution

Extraction properties of the solvent combination MeTHF/THF and toluene/THF (according to the present invention) were compared to those of the combination EtOAc/THF (comparative). The experiments were carried out with an aqueous solution containing 150 g/L NaCl. No peptides were present in the systems of the present example.

The volume ratios were as follows:

NMP:EtOAc:THF:NaCl solution=1:3:3:3

NMP:MeTHF:THF:NaCl solution=1:3:3:3

NMP:toluene:THF:NaCl solution=1:3:3:3

Fraction of NMP in the aqueous layer was determined by GC. The results of the experiments are summarized in Table 3 below.

TABLE 3
Extraction of NMP in the biphasic system NMP/solvent
1/solvent 2/NaCl solution (150 g/L NaCl).

| Solvent combination | Fraction of NMP in aqueous layer |
|---|---|
| EtOAc/THF | 0.857 |
| MeTHF/THF | 0.881 |
| toluene/THF | 0.911 |

As can be noticed from Table 3 above extractions with combinations MeTHF/THF and toluene/THF lead to a higher fraction of NMP in the aqueous layer than an extraction using EtOAc/THF. Accordingly, the NMP content in the organic layer after an extraction with MeTHF/THF or toluene/THF was lower than after an extraction with EtOAc/THF.

### Example 1

### Use of a Continuous LPPS Upon Usage of Fmoc as a Protecting Group for the Synthesis of H-Phe-Ile-Glu(OtBu)-Trp(Boc)-Leu-Lys(Boc)-Asn(Trt)-Gly-Pro-Thr(tBu)-Gly-Ser(tBu)-NH₂

### Example 1.1 LPPS

Fmoc-Phe-Ile-Glu(OtBu)-Trp(Boc)-Leu-Lys(Boc)-Asn(Trt)-Gly-OH (0.5 g, 0.28 mmol), H-Pro-Thr(tBu)-Gly-Ser(tBu)-NH₂ (0.15 g, 0.32 mmol) and HOBt (0.044 g, 0.28 mmol) were combined in NMP (2.5 mL) at 20° C. The mixture was stirred for 10 min at room temperature until all solids were dissolved, then cooled to 0° C. TBTU (0.093 g, 0.28 mmol), followed by DIPEA (46 μL, 0.28 mmol) was added and the reaction mixture was stirred at this temperature. After 2 h the reaction was complete as determined by HPLC. The reaction progress was monitored by the following method: 5 μL sample of the reaction mixture, diluted 50 fold in NMP, were analysed according to method MIH-009-3TG9 described above.

### Example 1.2 Fmoc Deprotection

To the solution prepared according to example 1.1 (4 mL) DEA (0.4 mL, 3.9 mmol) was added at room temperature. After completion of the Fmoc cleavage, as determined by HPLC, the volatiles were eliminated by co-evaporations with ACN (3×1 mL) at 30° C. and 60 mbar. The reaction progress was monitored by the following method: 5 μL sample of the reaction mixture, diluted 50 fold in NMP, were analysed according to method MIH-009-3TG9 described above.

### Example 1.3 Extraction with MeTHF/THF and Isolation

The solution prepared according to example 1.2 (4 mL) was combined with MeTHF (12 mL), THF (8 mL) and an aqueous solution containing 100 g/L NaCl and 25 g/L Na₂CO₃ (20 mL). After a thorough mixing and phase separation (approx. 4 min), the lower aqueous layer was removed. The peptide solution was further cleaned up by addition of THF (8 mL) and of an aqueous solution containing 100 g/L NaCl and 25 g/L Na₂CO₃ (20 mL). After a thorough mixing and a layer separation, the lower layer was removed. The organic layer was evaporated at 30° C., 60 mbar to a residual volume of ca. 4 mL. MeTHF and THF were removed by four co-evaporations with ACN (4×10 mL) to initiate the peptide precipitation. The process of peptide precipitation was completed by addition of ACN (10 mL) and DIPE (30 mL) to the residue of the fourth co-evaporation (4 mL). The solid was separated by filtration, washed with DIPE (3×10 mL) and dried under reduced pressure.

The present example demonstrates that the peptide precipitation can take place during evaporation of the organic layer and the precipitated peptide can be easily separated by filtration. In the presence of DMF or NMP, formation of such peptide precipitate would not be possible.

### Example 2

### Extraction of Boc-His(Trt)-Gly-Glu(OtBu)-Gly-Thr(tBu)-Phe-Thr(tBu)-Ser(tBu)-Asp(OtBu)-Leu-Ser(tBu)-Lys(Boc)-Gln(Trt)-Met-Glu(tBu)-Glu(tBu)-Glu(tBu)-Ala-Val-Arg(Pbf)-Leu-Phe-Ile-Glu(OtBu)-Trp(Boc)-Leu-Lys(Boc)-Asn(Trt)-Gly-Gly-Pro-Ser(tBu)-Ser(tBu)-Gly-Ala-Pro-Pro-Pro-Ser(tBu)-NH₂ from the Reaction Mixture

Boc-His(Trt)-Gly-Glu(OtBu)-Gly-Thr(tBu)-Phe-Thr(tBu)-Ser(tBu)-Asp(OtBu)-Leu-OH (6.94 g, 4.11 mmol), H-Ser(tBu)-Lys(Boc)-Gln(Trt)-Met-Glu(tBu)-Glu(tBu)-Glu(tBu)-Ala-Val-Arg(Pbf)-Leu-Phe-Ile-Glu(OtBu)-Trp(Boc)-Leu-Lys(Boc)-Asn(Trt)-Gly-Gly-Pro-Ser(tBu)-Ser(tBu)-Gly-Ala-Pro-Pro-Pro-Ser(tBu)-NH₂ (20 g, 4.32 mmol) and HOBt (0.63 g, 4.11 mmol) were combined in NMP (210 mL) at 20° C. The mixture was stirred for 10 min at room temperature until all solids were dissolved, then cooled to 0° C. TOTU (2.7 g, 8.22 mmol), followed by DIPEA (6 mL, 42 mmol) was added and the reaction mixture was stirred at this temperature. After 2 h the reaction was complete as determined by HPLC. The reaction progress was monitored by the following method: 5 μL sample of the reaction mixture, diluted 50 fold in NMP, were analysed according to method MIH-009-397TG15.

The obtained reaction mixture was divided in equal samples (sample volume: 5 mL) and used directly for the extractions tests #1-17. In each case, a sample of the reaction mixture (5 mL) was mixed with different organic solvents as summarised in Table 4, and then extracted with 15 mL of 20% aqueous solution of NaCl. These experimental conditions were compared for the phase separation (decantation) time and the yield of peptide extraction (ratio of the peptide in the organic layer).

In tests #1, 2, 7, 8, 15-17 a separation between two clear layers was rapidly observed (in less than 2 minutes). The two layers were separated and the peptide content in each layer was determined by HPLC.

In tests #3-6, 9-14 the extraction resulted in an opaque mixture. After several minutes (more than 60 minutes), the system began to separate but a thick layer of peptide gel formed between the aqueous layer and the organic layer. A clear separation of the layers was never observed. However, after 120 minutes, the aqueous layer was removed from the decantation vessel. The separation of the peptide gel from the organic layer was practically impossible (the density difference was too low). The peptide gel and the organic layer were dissolved with NMP and the peptide content was determined by HPLC. The yield of peptide extraction shown in Table 6 is thus more indicative of the quality of the decantation between the peptide gel than a real portioning between the aqueous layer and the organic layer.

The volume ratios of the components employed for the extractions tests #1-17 and observations are summarised in Table 4 below.

TABLE 4
Extraction of Boc-His(Trt)-Gly-Glu(OtBu)-Gly-Thr(tBu)-Phe-Thr(tBu)-Ser(tBu)-
Asp(OtBu)-Leu-Ser(tBu)-Lys(Boc)-Gln(Trt)-Met-Glu(tBu)-Glu(tBu)-Glu(tBu)-Ala-Val-
Arg(Pbf)-Leu-Phe-Ile-Glu(OtBu)-Trp(Boc)-Leu-Lys(Boc)-Asn(Trt)-Gly-Gly-Pro-Ser(tBu)-
Ser(tBu)-Gly-Ala-Pro-Pro-Pro-Ser(tBu)-NH₂

| | | | | | | Decantation | Peptide |
|---|---|---|---|---|---|---|---|
| | Vol DCM | Vol EtOAc | Vol MeTHF | Vol THF | Vol ACN | time | extraction |
| Test # | (mL) | (mL) | (mL) | (mL) | (mL) | (min) | yield (%) |
| 1 | 15 | 0 | 0 | 0 | 0 | <2 | 98.8 |
| 2 | 30 | 0 | 0 | 0 | 0 | <2 | 98.9 |
| 3 | 0 | 15 | 0 | 0 | 0 | 120 | 70.3* |
| 4 | 0 | 30 | 0 | 0 | 0 | 120 | 95.2* |
| 5 | 0 | 0 | 15 | 0 | 0 | 120 | 85.2* |
| 6 | 0 | 0 | 30 | 0 | 0 | 120 | 97.8* |
| 7 | 15 | 15 | 0 | 0 | 0 | <2 | 52.1 |
| 8 | 15 | 0 | 0 | 0 | 7.5 | <2 | 69.0 |
| 9 | 0 | 15 | 15 | 0 | 0 | 120 | 95.3* |
| 10 | 0 | 15 | 0 | 0 | 5 | 120 | 53.3* |
| 11 | 0 | 15 | 0 | 0 | 7.5 | 120 | 97.5* |
| 12 | 0 | 15 | 0 | 0 | 10 | 120 | 96.7* |
| 13 | 0 | 15 | 0 | 0 | 15 | 120 | 88.5* |
| 14 | 0 | 0 | 15 | 0 | 5 | 120 | 89.6* |
| 15 | 0 | 0 | 15 | 0 | 7.5 | <2 | 99.3 |
| 16 | 0 | 0 | 15 | 0 | 10 | <2 | 98.7 |
| 17 | 0 | 0 | 15 | 0 | 15 | <2 | 98.9 |

*peptide in the gel phase was included in the extraction yield

In tests #4, 6 and 11 the peptide was poorly solubilised in the organic layer; it was found as a gel that slowly settled between the organic layer and the aqueous layer.

### Results

In extractions with neat DCM (tests #1 and 2) a quick phase separation and a high peptide extraction yields were observed. However, as shown in example c) (Table 3) and comparative example 3.1 (Table 5), extractions with neat DCM result in a high content of the polar aprotic solvent in the organic layer. As a consequence, subsequent precipitation of the extracted peptide becomes difficult. Accordingly, implementation of peptide extractions with neat DCM suffers from serious drawbacks.

Extractions with neat MeTHF (tests #5 and 6) showed a higher peptide extraction yield than extractions with neat EtOAc (tests #3 and 4).

Extraction properties of mixtures MeTHF/ACN were investigated in tests #14-17. The phase separation times observed in tests #14-17 were shorter and the peptide extraction yields were higher in comparison to extractions with neat MeTHF (tests #5 and 6). A comparison between the results of extractions with MeTHF/ACN (tests #14-17) and extractions with EtOAc/ACN (tests #10-13) reveals that MeTHF/ACN mixtures have better extraction properties than the corresponding EtOAc/ACN mixtures, in particular, extractions with MeTHF/ACN mixtures led to shorter phase separation times and higher peptide extraction yields.

### Example 3

### Use of Continuous LPPS for the Coupling of Two Peptides and Boc Cleavage without Precipitation of the Intermediates. Preparation of Boc-Gly-Gly-Gly-Gly-Gly-Ser(Bzl)-Phe-Pro-Ile-Leu-Pro-Pro-Glu(OBzl)-Glu(OBzl)-Tyr-Leu(OBzl)

### Example 3.1 Boc-Pro-Ile-Leu-Pro-Pro-Glu(OBzl)-Glu(OBzl)-Tyr-Leu(OBzl)

Boc-Pro-Ile-Leu-Pro-Pro-OH (3.5 g, 5.5 mmol) and H-Glu(OBzl)-Glu(OBzl)-Tyr-Leu(OBzl) (5.0 g, 5.5 mmol) were dissolved in DMF (25 mL) at 20° C. The resulting mixture was cooled to −8° C. then HOBt-H₂O (0.88 g, 5.75 mmol), EDC-HCl (1.21 g, 6.31 mmol) were added and the reaction temperature was maintained in the range from −4° C. to −8° C. until a complete conversion was confirmed by a HPLC measurement. The reaction progress was monitored by the following method: 5 μL sample of the reaction mixture was diluted 50 fold in acetic acid:water (9:1) and analysed according to method MIH-009-2TG11 described above.

To a reaction mixture prepared above, MeTHF (90 mL) was added and the reaction mixture was successively extracted with:
1) aqueous solution containing 20 g/L NaCl (90 mL)
2) aqueous solution containing 20 g/L NaCl (90 mL)
3) aqueous solution containing 20 g/L NaCl and 50 g/L NaHCO₃ (90 mL)
4) aqueous solution containing 20 g/L NaCl and 50 g/L KHSO₄ (90 mL)
5) aqueous solution containing 20 g/L NaCl (90 mL).

The organic layer was then evaporated at 30° C. under reduced pressure.

### Comparative Example 3.1 Extraction of Boc-Pro-Ile-Leu-Pro-Pro-Glu(OBzl)-Glu(OBzl)-Tyr(Bzl)-Leu-OBzl

Boc-Pro-Ile-Leu-Pro-Pro-OH (3.5 g), H-Glu(OBzl)-Glu(OBzl)-Tyr(Bzl)-Leu-OBzl (5.0 g) and HOBt (0.88 g) were dissolved in DMF (20 mL). The coupling reaction was performed overnight under stirring at −6° C. to 0° C. with EDC HCl (1.2 g) and TEA (1.5 mL). Completion of the reaction was verified by HPLC (method MIH-009-2TG11). The reaction mixture was filtered to remove insoluble salts. Samples of 1 mL of reaction mixture were mixed with organic solvents as shown in Table 5 below and were then extracted with 3 mL of aqueous solution of NaCl (15% w/v) and Na₂CO₃ (2.5% w/v).

A rapid separation between the two clear layers was observed in all extraction tests. The DMF content in the organic layer was determined by GC.

TABLE 5
Extraction of Boc-Pro-Ile-Leu-Pro-Pro-Glu(OBzl)-Glu(OBzl)-
Tyr(Bzl)-Leu-OBzl

| | | Vol | Vol | | | |
|---|---|---|---|---|---|---|
| | Vol DCM | EtOAc | MeTHF | Vol THF | Vol ACN | DMF |
| Test # | (mL) | (mL) | (mL) | (mL) | (mL) | % (v/v) |
| 1 | 3 | 0 | 0 | 0 | 0 | 5.8 |
| 2 | 6 | 0 | 0 | 0 | 0 | 5.3 |
| 3 | 0 | 3 | 0 | 0 | 0 | 2.0 |
| 4 | 0 | 6 | 0 | 0 | 0 | 2.3 |
| 5 | 0 | 0 | 3 | 0 | 0 | 1.9 |
| 6 | 0 | 0 | 6 | 0 | 0 | 1.7 |
| 7 | 3 | 3 | 0 | 0 | 0 | 3.5 |
| 8 | 0 | 0 | 3 | 3 | 0 | 2.4 |
| 9 | 0 | 3 | 3 | 0 | 0 | 2.3 |
| 10 | 0 | 3 | 0 | 3 | 0 | 2.5 |
| 11 | 0 | 0 | 3 | 0 | 3 | 2.1 |

### Results

Extractions with neat MeTHF (tests #5 and 6) led to a lower DMF content in the organic layer than extractions with neat DCM (tests #1 and 2) or neat EtOAc (tests #3 and 4). Furthermore, extractions with solvent mixtures containing MeTHF (tests #8, 9 and 11) provided a lower DMF content in the organic layer than extraction with the mixture EtOAc/DCM (test #7) or EtOAc/THF (test #10).

### Example 3.2 Removal of the Boc Protecting Group. H-Pro-Ile-Leu-Pro-Pro-Glu(OBzl)-Glu(OBzl)-Tyr-Leu(OBzl)

Boc cleavage was performed by addition of toluene (20 mL), phenol (0.25 g) and TFA (16 mL) to the material obtained in example 3.1 at 15° C. After reaction completion, as determined by HPLC, the reaction mixture was evaporated at 30° C. under reduced pressure. The reaction progress was monitored by the following method: 5 μL sample of the reaction mixture, diluted 20 fold in ACN, were analysed according to method MIH-009-2TG11 described above.

Volatiles were further removed by subsequent co-evaporations with toluene (2×20 mL) at 30° C. under reduced pressure. MeTHF (50 mL) was added to the residue of evaporation and the organic solution was extracted six times with an aqueous solution containing 20 g/L NaCl (6×50 mL). The organic layer was evaporated at 30° C. under reduced pressure.

### Comparative Example 3.2 Influence of Residual DMF on the Removal of the Boc Protecting Group. H-Pro-Ile-Leu-Pro-Pro-Glu(OBzl)-Glu(OBzl)-Tyr(Bzl)-Leu-OBzl

The products from tests #1, 3 and 5 of comparative example 3.1 were further processed. The organic layers were separated and the solvents were exchanged by three co-evaporations with toluene (bath temperature=40° C., pressure=50 mbar). After the volatile solvents were completely evaporated, toluene (4 mL) and phenol (0.05 g) were added to the residues of evaporation. Boc cleavages were performed at 0° C. by addition of 3.5 mL TFA. The reactions were monitored by HPLC (method MIH-009-2TG11).

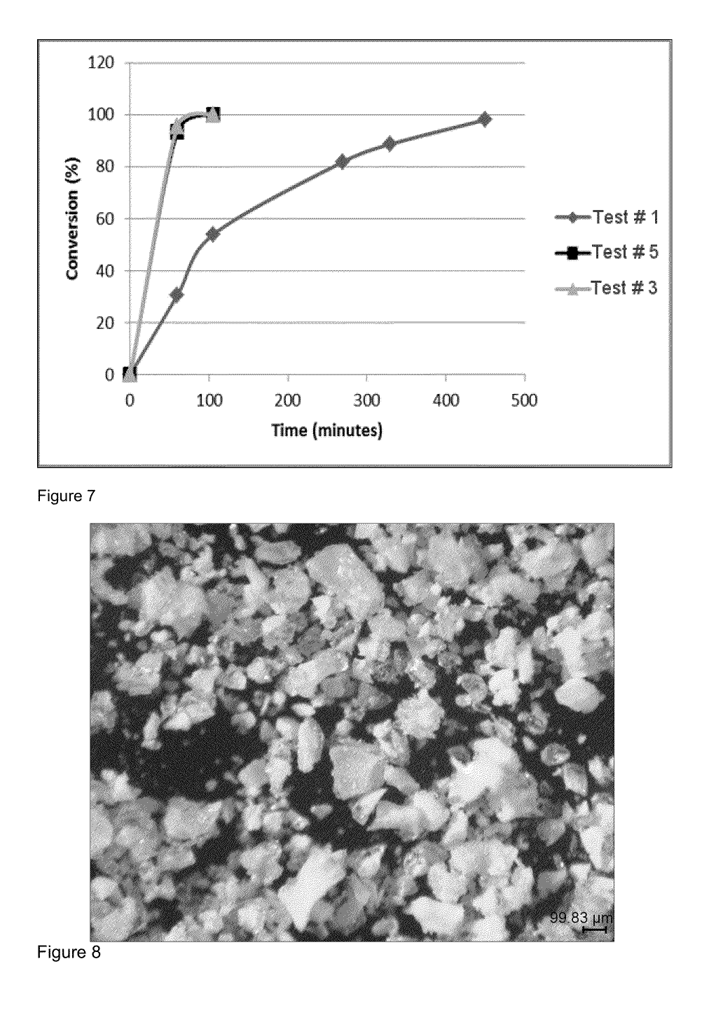

The obtained results are summarised in Table 6 and graphically presented in FIG. 7.

TABLE 6
Deprotection of Boc-Pro-Ile-Leu-Pro-Pro-Glu(OBzl)-
Glu(OBzl)-Tyr(Bzl)-Leu-OBzl

| | Conversion (%) | | |
|---|---|---|---|
| Time (min) | Test # 1 | Test # 3 | Test # 5 |
| 0 | 0 | 0 | 0 |
| 60 | 30.6 | 95.6 | 93.3 |
| 105 | 53.7 | 99.9 | 99.9 |
| 270 | 81.8 | | |
| 330 | 88.6 | | |
| 450 | 98 | | |

### Results

Traces of DMF in the materials obtained in comparative example 5.1 significantly inhibit the removal of Boc protective group. Thus, Boc cleavage of the material obtained by extraction with DCM was significantly slower than in the case of materials obtained by extraction with EtOAc and MeTHF. In this particular case no significant difference between materials obtained by extraction EtOAc and MeTHF was observed.

### Example 3.3 Coupling of Boc-Phe-OH with H-Pro-Ile-Leu-Pro-Pro-Glu(OBzl)-Glu(OBzl)-Tyr-Leu(OBzl) by Using LPPS without Precipitation of the Intermediate

Boc-Phe-OH (1.53 g, 5.8 mmol) was dissolved in DMF (25 mL) at 20° C. and added to the reaction mixture obtained in example 3.2. HOBt.H₂O (0.89 g, 5.8 mmol) and EDC.HCl (1.2 g, 6.3 mmol) were added thereto, and the reaction mixture was cooled to 5° C. The reaction mixture was kept at this temperature until a complete conversion was confirmed by HPLC. The reaction progress was monitored by the following method: 5 μL sample of the reaction mixture was diluted 50 fold in acetic acid:water (9:1) and analysed according to method MIH-009-2TG11 described above.

Then MeTHF (90 mL) was added and the reaction mixture was successively extracted with:
1) aqueous solution containing 50 g/L NaCl (90 mL)
2) aqueous solution containing 50 g/L NaCl (90 mL)
3) aqueous solution containing 20 g/L NaCl and 50 g/L NaHCO₃ (90 mL)
4) aqueous solution containing 20 g/L NaCl and 50 g/L KHSO₄ (90 mL)
5) aqueous solution containing 50 g/L NaCl (90 mL)
6) aqueous solution containing 50 g/L NaCl (90 mL).

The organic layer was then evaporated under reduced pressure at 35° C.

The process of example 3.2 was then applied to the obtained material with the only difference that the residue was extracted with aqueous NaCl solution seven times instead of six.

### Example 3.4 Coupling of Boc-Ser(Bzl)-OH and H-Phe-Pro-Ile-Leu-Pro-Pro-Glu(OBzl)-Glu(OBzl)-Tyr-Leu(OBzl)

Boc-Ser(Bzl)-OH (1.62 g, 5.5 mmol) was coupled to the H-Phe-Pro-Ile-Leu-Pro-Pro-Glu(OBzl)-Glu(OBzl)-Tyr-Leu(OBzl) peptide prepared according to example 3.2, using the procedure described therein.

### a) Extraction and Precipitation in DIPE

25 mL of the reaction mixture resulting from example 3.4 and containing 5 g Boc-Ser(Bzl)-Phe-Pro-Ile-Leu-Pro-Pro-Glu(OBzl)-Glu(OBzl)-Tyr(Bzl)-Leu(OBzl) were combined with MeTHF (75 mL) and an aqueous solution containing 100 g/L NaCl (75 mL). After a thorough mixing and phase separation (approx. 4 min) the lower aqueous layer was removed. The upper organic layer was further extracted three times with an aqueous solution containing 100 g/L NaCl (3×75 mL). The organic layer was finally isolated and partially evaporated at 30° C., 60 mbar to a residual volume of 10 mL. The partially evaporated organic layer was added dropwise under stirring into DIPE (250 mL) at 0° C. whereby the precipitation of the peptide took place. The resulting mixture was transferred into a 2.7 cm diameter filtration column equipped with a 20 μm pore size filter. The filtration was carried out under a pressure of 50 mbar. The total mother liquor of precipitation (260 mL) was filtered in 3 minutes and 45 seconds. The cake heights after filtration was 3.5 cm giving a filterability coefficient K=848. The solids were collected and dried under reduced pressure. 4.5 g of the peptide was isolated as a solid material.

An image of the isolated peptide is shown as FIG. 8 (40× enlargement).

The aqueous layer resulting from the extraction process and the mother liquors of precipitation were analysed by HPLC. The amount of the peptide detected therein was below 0.5 wt.-% of the total amount of the peptide present in 25 mL of the reaction mixture resulting from example 3.4.

### b) Comparative Example

### Influence of DMF Addition to the Mother Liquors of precipitation

The procedure of extraction and precipitation was performed as described under a) above but DMF (2.5 mL) was added to the precipitation mixture before the filtration of the peptide was carried out. The solid precipitate immediately turned into a gum-like solid that was not filterable.

### c) Comparative Example

### Direct Precipitation in DIPE

25 mL of the reaction mixture obtained in example 3.4, containing 5 g Boc-Ser(Bzl)-Phe-Pro-Ile-Leu-Pro-Pro-Glu(OBzl)-Glu(OBzl)-Tyr(Bzl)-Leu(OBzl) were added dropwise into DIPE (250 mL) under stirring at 0° C. for precipitation. The peptide precipitated in the form of a sticky gum-like solid. After decantation the supernatant was pumped off and replaced with a second batch of DIPE (250 mL). The resulting mixture was stirred for one hour in order to de-aggregate the sticky gum-like solid. After decantation the supernatant was replaced again with a third batch of DIPE (250 mL). The mixture was stirred again for one hour and it was finally transferred into the filtration column. However, a large part of the solid was still in the form of a sticky gum-like solid that was left stuck onto the precipitation vessel and therefore could not be transferred. The mother liquors were filtered in 2 min 30 sec, yielding a 1.75 cm high cake. This gave a filtration coefficient K=636. The collected solids were dried under reduced pressure.

2.45 g of the peptide were isolated.

### d) Comparative Example

### Direct Precipitation in Water

25 mL of the reaction mixture resulting from example 3.4 and containing 5 g Boc-Ser(Bzl)-Phe-Pro-Ile-Leu-Pro-Pro-Glu(OBzl)-Glu(OBzl)-Tyr(Bzl)-Leu(OBzl) were added dropwise into water (250 mL) under stirring at 0° C. for precipitation. This yielded a very thin precipitate that was subsequently transferred into the filtration column. The filtration rate was very low (<3 mL/h), a considerable amount of precipitate went through the filter in the beginning of the filtration and the filter was definitely clogged after about 65 min. Moreover, there was no clear decantation of the precipitate. Thus, it was not possible to collect the obtained precipitate.

### Example 3.5 Boc Cleavage of Boc-Ser(Bzl)-Phe-Pro-Ile-Leu-Pro-Pro-Glu(OBzl)-Glu(OBzl)-Tyr-Leu(OBzl)

Boc-Ser(Bzl)-Phe-Pro-Ile-Leu-Pro-Pro-Glu(OBzl)-Glu(OBzl)-Tyr(Bzl)-Leu(OBzl) (5 g) were put in a mixture of toluene (20 mL), phenol (0.2 g) and TFA (16 mL). After reaction completion as determined by HPLC (5 μL of the reaction, diluted 30 fold in acetonitrile, were analysed according to HPLC method MIH-009-2TG11), the reaction mixture was evaporated under reduced pressure and a residual oil was obtained. The residual TFA was further removed by two co-evaporations with toluene (2×30 mL). MeTHF (50 mL) was added to the resulting residue of co-evaporations and this mixture was extracted three times with an aqueous solution containing NaCl at 100 g/L (3×50 mL). The obtained organic layer was separated and evaporated under reduced pressure at 35° C.

### Example 3.6 Coupling of Boc-Gly-Gly-Gly-Gly-OH with H-Ser(Bzl)-Phe-Pro-Ile-Leu-Pro-Pro-Glu(OBzl)-Glu(OBzl)-Tyr-Leu(OBzl) and Extraction of the Product

Boc-Gly-Gly-Gly-Gly-OH (1.27 g, 2.8 mmol), H-Ser(Bzl)-Phe-Pro-Ile-Leu-Pro-Pro-Glu(OBzl)-Glu(OBzl)-Tyr(Bzl)-Leu(OBzl) (5.0 g, 2.7 mmol) and HOBt.H₂O (0.43 g, 2.8 mmol) were dissolved in DMF (25 mL) at 20° C. and the obtained solution was added to the reaction mixture obtained in example 3.5 above. The temperature of the reaction mixture was adjusted to 6±2° C., and EDC.HCl (0.6 g, 3.1 mmol) was added thereto. The reaction mixture was kept at this temperature until a complete conversion was confirmed by HPLC. The reaction progress was monitored by the following method: 3 μL sample of the reaction mixture, diluted 50 fold in acetic acid:water (9:1), were analysed according to method MIH-009-2TG11 described above.

Then, MeTHF (90 mL) and THF (30 mL) were added and the mixture was successively extracted with:
1) aqueous solution containing 100 g/L NaCl (100 mL)
2) aqueous solution containing 100 g/L NaCl and 25 g/L NaHCO₃ (100 mL)
3) aqueous solution containing 100 g/L NaCl (100 mL)
4) aqueous solution containing 100 g/L NaCl (100 mL).

The obtained organic layer was then evaporated under reduced pressure at 35° C.

### Example 3.7 Boc Cleavage

Toluene (20 mL), phenol (0.2 g) and TFA (16 mL) were added to the residue of evaporation obtained in the example 3.6 above. After reaction completion as determined by HPLC (5 μL of the reaction, diluted 30 fold in acetonitrile, were analysed according to HPLC method MIH-009-2TG11), the reaction mixture was evaporated under reduced pressure whereby a residual oil was obtained. The residual TFA was removed by two subsequent co-evaporations with toluene (2×3 mL). MeTHF (60 mL) and THF (50 mL) were added to the residue of evaporation and the resulting solution was extracted three times with an aqueous solution containing 100 g/L NaCl (3×100 mL). The obtained organic layer was evaporated under reduced pressure at 35° C.

### Example 3.8 Coupling of Boc-Gly-OH with H-Gly-Gly-Gly-Gly-Ser(Bzl)-Phe-Pro-Ile-Leu-Pro-Pro-Glu(OBzl)-Glu(OBzl)-Tyr-Leu(OBzl)

DMF (25 mL) was added to the residue of evaporation obtained in example 3.7 above. Boc-Gly-OH (0.5 g, 2.8 mmol), HOBt.H₂O (0.43 g, 2.8 mmol) and EDC.HCl (0.6 g, 3.1 mmol) were added to the resulting mixture at 6±2° C. The reaction mixture was kept at this temperature until a complete conversion was confirmed by HPLC. The reaction progress was monitored by the following method: 3 μL sample of the reaction mixture, diluted 50 fold in acetic acid:water (9:1), were analysed according to method MIH-009-2TG11 described above.

Subsequently, MeTHF (90 mL) and THF (30 mL) were added and the resulting mixture was successively extracted with:
1) aqueous solution containing 100 g/L NaCl (100 mL)
2) aqueous solution containing 100 g/L NaCl and 25 g/L NaHCO₃ (100 mL)
3) aqueous solution containing 100 g/L NaCl (100 mL)
4) aqueous solution containing 100 g/L NaCl (100 mL)
5) aqueous solution containing 100 g/L NaCl (100 mL).

The resulting organic layer was evaporated under reduced pressure at 35° C.

### Example 4 Coupling of Boc-MeLeu-OH and HCl.Ala-OMe

HCl.Ala-OMe (4.6 g, 33.1 mmol) was dissolved in DMF (35 mL) at 20° C. The obtained solution was cooled to −5° C. and Boc-MeLeu-OH (7.1 g, 28.8 mmol), HOBt (3.9 g, 0.29 mmol) and EDC.HCl (5.5 g, 28.8 mmol) were added thereto. The reaction mixture was kept at −5° C. until completion of the reaction as monitored by the following method: 5 μL sample of the reaction mixture, diluted 10 fold in acetic acid in methanol, were analysed according to method MIH-009-025TG3 described above.

After completion of the reaction, MeTHF (130 mL) was added and the mixture was extracted:
1) once with water (130 mL)
2) once with aqueous solution containing 50 g/L NaCl (40 mL)
3) three times with aqueous solution containing 10 g/L KHSO₄ (40 mL).

Subsequently, n-heptane (10 mL) was added to the organic layer and the combined layer was extracted:
1) once with aqueous solution containing 50 g/L NaHCO₃ (25 mL)
2) once with water (25 mL).

The organic layer was then evaporated under reduced pressure. n-Heptane (140 mL) was added to the residue and the mixture was again evaporated under reduced pressure, whereby the crystallisation of the peptide took place. After 18 hours, the solids were separated by filtration and rinsed twice with n-heptane.

The collected product was re-dissolved in n-heptane (45 mL) at 40° C. and left overnight for re-crystallisation.

Because HCl.H-Ala-OMe is highly hydrolysable, it usually contains some HCl.H-Ala-OH. Therefore, the material isolated after the peptide coupling reaction usually contains Boc-MeLeu-Ala-Ala-OMe as an impurity. In general, impurities having a double Ala in the sequence are known to be difficult to remove by chromatography after the complete peptide synthesis was carried out.

The re-crystallisation employed in the present example allows decreasing of the amount of Boc-MeLeu-Ala-Ala-OMe, which is present in the isolated peptide as an impurity, from 1.2 mol-% to 0.2 mol.-%. This re-crystallisation is only possible in the absence of DMF.

### Example 5 Use of Continuous LPPS for a Stepwise Peptide Assembly Without Precipitation of the Intermediates. Preparation of H-Pro-Ala-Gly-Phe-Ser(tBu)-xantheneamide

### Example 5.1 Coupling Fmoc-Phe-OH to H-Ser(tBu)-xantheneamide

H-Ser(tBu)-xantheneamide (2.5 g, 7.7 mmol) and Fmoc-Phe-OH (3.0 g, 7.7 mmol) were dissolved in NMP (20 mL) at 20° C. TBTU (2.6 g, 8.1 mmol) and TEA (2 mL) were added and the reaction progress was monitored by the following method: 1 μL sample of the reaction mixture, diluted 50 fold in DMF, was analysed according to method MIH-009-RTTG 1.

After completion of the reaction, MeTHF (75 mL) and THF (25 mL) were added to the reaction mixture. The obtained organic layer was extracted with an aqueous solution (75 mL) containing 100 g/L NaCl. After vigorous stirring of the resulting mixture and separation of the organic layer, the organic layer was evaporated under reduced pressure. The peptide was precipitated by addition of acetonitrile (100 mL) to the residue of evaporation. The resulting solid was separated by filtration and dried under reduced pressure.

### Example 5.2 Fmoc Cleavage of Fmoc-Phe-Ser(tBu)-xantheneamide

Fmoc-Phe-Ser(tBu)-xantheneamide (2 g) obtained in example 5.1 was dissolved in a mixture of NMP (15 mL) and TAEA (2 mL). After the reaction completion, as determined by the method specified in example 5.1 above, MeTHF (100 mL) and THF (100 mL) were added to the reaction mixture. It was then extracted:
1) three times with aqueous solution containing 100 g/L NaHCO₃ (30 mL)
2) five times with aqueous solution containing 10 g/L KHSO₄ (30 mL)
3) five times with aqueous solution containing 20 g/L NaHCO₃ (30 mL)
4) twice with aqueous solution containing 150 g/L NaHCO₃ (30 mL).

NMP (30 mL) was added and the resulting organic layer was evaporated under reduced pressure.

### Example 5.3 Coupling of Fmoc-Gly-OH with H-Phe-Ser(tBu)-xantheneamide

Fmoc-Gly-OH (0.92 g, 3.1 mmol), TBTU (1.0 g, 3.1 mmol) and TEA (0.9 mL) were added to the residue of evaporation obtained in example 5.2. The reaction completion was verified as specified in example 5.1 above.

### Example 5.4 Fmoc Cleavage of Fmoc-Gly-Phe-Ser(tBu)-xantheneamide

TAEA (3 mL) was added to the reaction mixture obtained in example 5.3. After a complete conversion was confirmed by the method specified in example 5.1 above, MeTHF (100 mL) was added to the reaction mixture. It was then extracted:
1) once with aqueous solution containing 100 g/L NaCl (100 mL)
2) four times with mixture of aqueous solution containing 100 g/L NaCl (21 mL) and NMP (3.7 mL)
3) once with aqueous solution containing 200 g/L NaCl (25 mL).

NMP (30 mL) was added and the resulting organic layer was evaporated under reduced pressure.

### Example 5.5 Coupling of Fmoc-Ala-OH to H-Gly-Phe-Ser(tBu)-xantheneamide

Fmoc-Ala-OH (0.97 g, 3.1 mmol), TBTU (1.0 g, 3.1 mmol) and TEA (0.8 mL) were added to the residue of evaporation obtained in example 5.4 above. The reaction completion was verified by the method specified in example 5.1 above.

### Example 5.6 Fmoc Cleavage of Fmoc-Ala-Gly-Phe-Ser(tBu)-xantheneamide

TAEA (3 mL) was added to the coupling reaction mixture obtained in example 5.5. After the reaction completion, as determined by the method specified in example 5.1 above, MeTHF (100 mL) was added to the reaction mixture. It was then extracted:
1) once with aqueous solution containing 100 g/L NaCl (100 mL)
2) four times with mixture of aqueous solution containing 100 g/L NaCl (21 mL) and NMP (4 mL)
3) once with aqueous solution containing 200 g/L NaCl (25 mL).

NMP (30 mL) was added and the resulting organic layer was evaporated under reduced pressure.

### Example 5.7 Coupling of Fmoc-Pro-OH with H-Ala-Gly-Phe-Ser(tBu)-xantheneamide

Fmoc-Pro-OH (1.05 g, 3.1 mmol), TBTU (1.0 g, 3.1 mmol) and TEA (0.8 mL) were added to the residue of evaporation obtained in example 5.6 above. The reaction completion was verified by the method specified in example 5.1.

### Example 5.8 Fmoc Cleavage of Fmoc-Pro-Ala-Gly-Phe-Ser(tBu)-xantheneamide

TAEA (3 mL) was added to the coupling reaction mixture obtained in example 5.7. After the reaction completion was verified by the method described in example 5.1 above, MeTHF (100 mL) was added to the reaction mixture. It was then extracted:
1) once with aqueous solution containing 100 g/L NaCl (100 mL)
2) four times with mixture of aqueous solution containing 100 g/L NaCl (42 mL) and NMP (8 mL)
3) once with aqueous solution containing 200 g/L NaCl (25 mL).

ACN (50 mL) was added to the obtained organic layer and the resulting mixture was evaporated under reduced pressure to initiate the peptide precipitation. After three further co-evaporations with ACN (3×30 mL), the obtained solid peptide was separated by filtration and dried under reduced pressure.

### Comparative Example 1 SPPS of H-Leu-Trp(Boc)-Val-Asn(Trt)-Ser(tBu)-NH₂ upon Usage of Sieber Resin and Fmoc and t-Bu as Protecting Groups

The SPPS was carried out manually on 10 mmol scale upon using Sieber resin (2.3 g) with loading of 0.61 meq/g. The materials consumed during the peptide synthesis are listed in Table 5 below.

The Sieber resin was swollen in DCM (20 mL) for 18 hours and then washed six times with DMF. The peptide was then assembled onto the resin, using the following procedure for each amino acid incorporation:
1. Fmoc cleavages: three treatments of 15 min each with mixture of piperidine/DMF (15 mL, v/v=2/8).
2. peptide-resin wash: six times with DMF (10 mL).
3. amino acid coupling: Fmoc-amino acid (2.1 mmol, 1.5 eq.) with PyBOP (2.1 mmol) in DMF (10 mL) and TEA (0.7 mL). The completeness of the reaction was verified by the Kaiser test.
4. peptide-resin wash: six times with DMF (10 mL).

After the final Fmoc cleavage, the resin was washed eight times with DMF (10 mL) and then six times with DCM (10 mL). The peptide was cleaved off the resin with four successive treatments with DCM/TFA (v/v=95/5) for 10 min. The resulting solutions were combined, evaporated under reduced pressure and precipitated in DIPE (20 mL). The obtained solids were dried under reduced pressure.

605 mg (gross yield=33%) of H-Leu-Trp(Boc)-Val-Asn(Trt)-Ser(tBu)-NH₂ were isolated and the purity of the product was determined to be 57%.

The values in the Table 7 below are given for a 10 mmol synthesis scale carried out by SPPS vs. LPPS.

TABLE 7
Materials consumed during the synthesis of H-Leu-
Trp(Boc)-Val-Asn(Trt)-Ser(tBu)—NH₂ according
to the methods of comparative example 1.
Comparative example 1

| | | |
|---|---|---|
| Sieber resin | 16.5 | g |
| DCM | 142.86 | mL |
| DMF | 8100.00 | mL |
| piperidine | 257.14 | mL |
| PyBOP | 39.29 | g |
| TEA | 21.43 | mL |
| Fmoc-Ser(tBu)-OH | 5.75 | g |
| Fmoc-Asn(Trt)-OH | 8.93 | g |
| Fmoc-Val-OH | 5.09 | g |
| Fmoc-Trp(Boc)-OH | 7.86 | g |
| Fmoc-Leu-OH | 5.36 | g |

In summary, the purity and the yield of the target peptide prepared in comparative example 1 were lower than usual values observed in a process for preparation of a peptide according to the present invention.

### Comparative Example 2

### Continuous LPPS of H-Leu-Trp(Boc)-Val-Asn(Trt)-Ser(tBu)-NH₂ According to Carpino's Method

### Comparative Example 2.1 LPPS of Fmoc-Asn(Trt)-Ser(tBu)-NH₂

H-Ser(tBu)-NH₂ (2.0 g, 12.5 mmol) and Fmoc-Asn(Trt)-OH (6.8 g, 11.3 mmol) were added to DCM (50.0 mL) at 20° C. The mixture was stirred for 15 min until the solids were completely dissolved and further cooled to 10° C. DCC (2.34 g, 11.3 mmol) and HOBt (1.74 g, 11.3 mmol) were added. The reaction was carried out at 10° C. and the conversion was complete after 14 h, as confirmed by HPLC. The reaction progress was monitored by the following method: 3 μL sample of the reaction mixture, diluted 50 fold in NMP, were analysed according to method MIH-009-RTTG1 described above.

### Comparative Example 2.2 Removal of the Fmoc Protecting Group and Isolation of Asn(Trt)-Ser(tBu)-NH₂

To a mixture prepared according to comparative example 2.1, TAEA (25 mL) was added and the reaction mixture was stirred at room temperature. The completion of the Fmoc cleavage was determined by HPLC, using the same method as in example 4.1.

The DCU was separated by filtration, whereby the filtration process took 6 min. The resulting filtrate was diluted with DCM to the total volume of 250 mL and subsequently extracted three times with an aqueous solution containing 100 g/L NaH₂PO₄ and Na₂HPO₄, pH 5.5 (100 mL).

### Comparative Example 2.3 Coupling of Fmoc-Val-OH with H-Asn(Trt)-Ser(tBu)-NH₂

The organic layer obtained in comparative example 2.2 was evaporated at 30° C. under reduced pressure to a residual volume of 80 mL.

Fmoc-Val-OH (3.85 g, 11.3 mmol), DCC (2.34 g, 11.3 mmol) and HOBt (1.74 g, 11.3 mmol) were added. The reaction was carried out at room temperature. After 18 h, Fmoc-Val-OH (0.77 g, 2.3 mmol), DCC (0.47 g, 2.3 mmol) and DCM (25 mL) were added in order to complete the reaction. The reaction progress was monitored by the following method: 3 μL sample of the reaction mixture, diluted 50 fold in DMF, were analysed according to method MIH-009-RTTG1 described above.

### Comparative Example 2.4 Removal of Fmoc Protecting Group. H-Val-Asn(Trt)-Ser(tBu)-NH₂

Fmoc cleavage was performed by addition of TAEA (25 mL) to the reaction mixture obtained in the comparative example 2.3. The completion of the reaction was verified by HPLC using the same method as in the comparative example 2.3.

DCU was separated by filtration and rinsed twice with DCM (2×25 mL). The obtained filtrates were combined and diluted to the total volume of 200 mL with DCM. The solution was extracted three times with an aqueous solution containing 100 g/L NaH₂PO₄ and Na₂HPO₄, pH 5.5 (3×100 mL).

The organic layer was evaporated under reduced pressure at 30° C. to a residual volume of 100 mL.

### Comparative Example 2.5 Coupling of Fmoc-Trp(Boc)-OH with H-Val-Asn(Trt)-Ser(tBu)-NH₂

Fmoc-Trp(Boc)-OH (6.0 g, 11.3 mmol), DCC (2.34 g, 11.3 mmol) and HOBt (1.74 g, 11.3 mmol) were added to the peptide solution obtained in comparative example 2.4. The coupling reaction was carried out at room temperature and the reaction time was 18 h. The completion of the reaction was confirmed by HPLC using the same method as in the comparative example 2.3.

### Comparative Example 2.6 Removal of the Fmoc Protecting Group. H-Trp(Boc)-Val-Asn(Trt)-Ser(tBu)-NH₂

Fmoc cleavage was performed by addition of TAEA (25 mL) to the reaction mixture obtained in the comparative example 2.5. The completion of the reaction was determined by HPLC using the same method as in the comparative example 2.3.

DCU was separated by filtration and rinsed twice with DCM (2×25 mL). The resulting filtrates were combined and diluted to the total volume of 200 mL with DCM. The solution was extracted three times with an aqueous solution containing 100 g/L NaH₂PO₄ and Na₂HPO₄, pH 5.5 (3×100 mL).

Since the organic layer became cloudy during the process of extraction, additional DCM was added to the organic layer, so that its volume was brought to 400 mL. Nevertheless, some undissolved product was present during the process of extraction. Therefore, the separation of the layers was difficult and some product was lost in the aqueous layer.

### Comparative Example 2.7 Coupling of Fmoc-Leu-OH with H-Trp(Boc)-Val-Asn(Trt)-Ser(tBu)-NH₂ by LPPS

Fmoc-Leu-OH (4.0 g, 11.3 mmol), DCC (2.34 g, 11.3 mmol) and HOBt (1.74 g, 11.3 mmol) were added to the peptide solution obtained in the comparative example 2.6. The coupling reaction was carried out at room temperature and the reaction time was 18 h. The completion of the reaction was determined by HPLC using the same method as in the comparative example 2.3.

### Comparative Example 2.8 Removal of the Fmoc Protecting Group. H-Leu-Trp(Boc)-Val-Asn(Trt)-Ser(tBu)-NH₂

Fmoc cleavage was performed by addition of TAEA (25 mL) to the reaction mixture obtained in the comparative example 2.5. The completion of the reaction was determined by HPLC using the same method as in the comparative example 2.3.

DCU was separated by filtration and rinsed twice with DCM (2×25 mL). The resulting filtrates were combined and diluted to the total volume of 200 mL with DCM. The solution was extracted three times with an aqueous solution containing 100 g/L NaH₂PO₄ and Na₂HPO₄, pH 5.5 (3×100 mL).

Since the organic layer became cloudy during the process of extraction, additional DCM was added to the organic layer, so that its volume was brought to 400 mL. Nevertheless, some undissolved product was present during the process of extraction. Therefore, the separation of the layers was difficult and some product was lost in the aqueous layer.

The resulting organic layer was evaporated at 30° C. under reduced pressure. The obtained residual oil was transferred into n-heptane (100 mL) for precipitation. The resulting solids were isolated by filtration, rinsed three times with n-heptane (3×10 mL) and dried under reduced pressure.

2.6 g of H-Leu-Trp(Boc)-Val-Asn(Trt)-Ser(tBu)-NH₂ (yield=31%) were isolated and the purity of the final product was 49%.

In summary, the synthetic method described by Carpino et al. showed several drawbacks. Because the solubility of the peptides H-Leu-Trp(Boc)-Val-Asn(Trt)-Ser(tBu)-NH₂ and H-Trp(Boc)-Val-Asn(Trt)-Ser(tBu)-NH₂ in DCM was not sufficient, a significant amount of these peptides precipitated during the process of extraction in the interface between the organic layer and the aqueous layer. Despite the volume of the organic layer was increased to 400 mL, the products were isolated in only a moderate yield. Furthermore, the separation of the resulting DCU by filtration was demonstrated to be time consuming.

1. A process for extraction of a peptide from a reaction mixture resulting from a peptide coupling reaction, the reaction mixture containing the peptide and a polar aprotic solvent selected from the group consisting of N,N-dimethylformamide, N,N-dimethylacetamide and N-methyl-2-pyrrolidone, whereby the process comprises a step a) and a step b),
step a) comprises the addition of a component a1), a component a2) and a component a3), whereby
component a1) is an organic solvent 1, the organic solvent 1 is selected from the group consisting of 2-methyltetrahydrofuran and toluene,
component a2) is water, and
component a3) is an organic solvent 2, the organic solvent 2 is selected from the group consisting of ethylacetate, isopropylacetate, acetonitrile, tetrahydrofuran and n-heptane to the reaction mixture, so that a biphasic system with an organic layer and an aqueous layer is obtained;
step b) comprises the separation of the organic layer containing the peptide from the aqueous layer,
whereby
the biphasic system obtained in step a) is characterised by the following volume ratios:
polar aprotic solvent:organic solvent 1 from 1:20 to 1:2;
polar aprotic solvent:organic solvent 2 from 1:5 to 30:1; and
polar aprotic solvent:water from 1:20 to 1:2.

2. The process of claim 1, whereby the biphasic system obtained in step a) is characterised by the following volume ratios:
polar aprotic solvent:organic solvent 1 from 1:6 to 1:3;
polar aprotic solvent:organic solvent 2 from 1:1 to 4:1; and
polar aprotic solvent:water from 1:5 to 1:3.

3. The process of claim 1, whereby the polar aprotic solvent is selected from the group consisting of N,N-dimethylformamide and N-methyl-2-pyrrolidone.

4. The process of claim 1, whereby the organic solvent 1 is 2-methyltetrahydrofuran.

5. The process of claim 1, whereby the organic solvent 2 is selected from the group consisting of acetonitrile and tetrahydrofuran.

6. The process of claim 1, whereby the component a2) contains at least one inorganic salt selected from the group consisting of sodium chloride, sodium hydrogensulfate, potassium hydrogensulfate, sodium hydrogencarbonate and sodium hydrogenphosphate.

7. The process of claim 1, whereby the pH value of the component a2) ranges from 5 to 8.

8. The process of claim 1, whereby a filtration of the biphasic system obtained in step a) is carried out before step b).

9. The process of claim 1, whereby step a) and step b) are carried out at a temperature of from 20° C. to 30° C.

10. A process for preparation of a peptide in liquid phase comprising a step aa), a step bb) and a step cc):
in step aa) a peptide coupling reaction is carried out in the polar aprotic solvent selected from the group consisting of N,N-dimethylformamide, N,N-dimethylacetamide and N-methyl-2-pyrrolidone, and in the presence of a coupling reagent;
in step bb) the resulting peptide is extracted according to a process according to claim 1; and
in step cc) at least a part of the organic layer obtained in step bb) is evaporated.

11. The process of claim 10, whereby the coupling reagent is selected from the group consisting of uronium salts, phosphonium salts of O-1H-benzotriazole and carbodiimide coupling reagents.

12. The process of claim 10, whereby a tertiary base is selected from the group consisting of N,N-diisopropylethylamine, triethylamine and N-methylmorpholine, and said tertiary base is present in the peptide coupling reaction of step aa).

13. The process of claim 10 comprising further a further step dd), a step ee) and a step ff), wherein
in step dd) the organic layer obtained in step cc) is combined with an organic solvent 3 selected from the group consisting of acetonitrile, diethyl ether, diisopropyl ether and toluene;
in step ee) at least a substantial part of the peptide is precipitated; and
in step ff) the precipitated peptide is separated by filtration.

14. The process of claim 10, whereby the organic layer obtained in step cc) is treated with trifluoroacetic acid in the case that a N-terminal protecting group of the peptide is a tert-butyloxycarbonyl protecting group, said tert-butyloxycarbonyl protecting group is removed by said treatment with trifluoroacetic acid.

15. The process of claim 10, whereby the reaction mixture resulting from the peptide coupling reaction and obtained in step aa) is treated with piperidine in the case that a N-terminal protecting group of the peptide is a fluorenyl-9-methoxycarbonyl protecting group, said fluorenyl-9-methoxycarbonyl protecting group is removed by said treatment with piperidine.

16. The process of claim 10, whereby the C-terminal carboxylic acid group of the peptide is protected as a 2-chlorophenyldiphenylmethylester or N-methyl-9H-xanthen-9-amide.
